SEQUENCE LISTING (6 sequences)

SEQ ID NO: 1 - DNA, 1548 bp, synthetic construct
  source 1..1548: mol_type = other DNA
atggattggacatggattctgttcctggtcgcagcagccacaagagtgcattccgatattcagatgacccagtccccttcaagcctgtccgcctctgtgggcgatagggtgaccatcagctgcagagccagcgagtccgtggacaactacggcatctccttcctgaattggtttcagcagaagcccggcaaggcccctaagctgctgatctatgcagcaagcaaccagcggtccggagtgccatctcgcttctctggaagcggatccggaaccgacttcaccctgacaatcagctccctgcagcccgaggacttcgccacatacttttgccagcagagcaaggaggtgccttggaccttcggccagggcacaaaggtggagatcaagaggggcggcggcggcagcggcggcggcggcagcggcggcggcggctctgaggtgcagctggtggagagcggcggcggcctggtgcagcctggcggctctctgaggctgtcttgtagcttctccggcttttctctgagcacctccggaatgggagtgggatggatcagacaggcaccaggcaagggcctggagtgggtggcccacatctggtgggacgatgacaagcggtacaaccccgccctgaagtctcgcttcaccctgagcgtggataggtccaagaacacactgtatctgcagatgaacagcctgagagccgaggacaccgccacatactattgcgtgcagatcaactacggcaattatcggtttgataattggggccacggcaccctggtgacagtgtctagcggcggcggcggctccgaagtccagctggtcgaaagcggcggcggcctggtccagccaggcggctctctgagactgagctgtgcagcatccggatactctttcaccggctatacaatgaattgggtgaggcaggcacctggcaagggcctggaatgggtggccctgatcaacccatacaagggcgtgagcacctataatcagaagttcaaggataggtttaccatctccgtggacaagtctaagaacacagcctacctgcaaatgaacagcttacgcgccgaggacacagccgtgtactattgcgccaggagcggctactatggcgattccgactggtattttgacgtgtggggccagggcaccctggtcacagtgtccagcggcggcggcggcagcggcggcggcggcagcggcggcggcggctcggacatccagatgacccagagcccaagctccctgtctgccagcgtcggcgatagggtcaccatcacatgtagagcctcccaggacatccggaactacctgaattggtatcagcagaagccaggcaaggcccccaagctgctgatctactatacaagcagactggagtccggcgtgccttctcggttctccggatctggaagcggaaccgattacaccctgacaatctctagcctgcagccagaggactttgccacatactattgtcagcaggggaatactctgccttggaccttcggacagggaacaaaagtggaaatcaaatcctca

SEQ ID NO: 2 - protein, 516 aa, synthetic construct
MDWTWILFLVAAATRVHSDIQMTQSPSSLSASVGDRVTISCRASESVDNYGISFLNWFQQKPGKAPKLLIYAASNQRSGVPSRFSGSGSGTDFTLTISSLQPEDFATYFCQQSKEVPWTFGQGTKVEIKRGGGGSGGGGSGGGGSEVQLVESGGGLVQPGGSLRLSCSFSGFSLSTSGMGVGWIRQAPGKGLEWVAHIWWDDDKRYNPALKSRFTLSVDRSKNTLYLQMNSLRAEDTATYYCVQINYGNYRFDNWGHGTLVTVSSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSS

SEQ ID NO: 3 - protein, 246 aa, synthetic construct
EVQLVESGGGLVQPGGSLRLSCSFSGFSLSTSGMGVGWIRQAPGKGLEWVAHIWWDDDKRYNPALKSRFTLSVDRSKNTLYLQMNSLRAEDTATYYCVQINYGNYRFDNWGHGTLVTVSSGGGGSGGGGSGGGGSDIQMTQSPSSLSASVGDRVTISCRASESVDNYGISFLNWFQQKPGKAPKLLIYAASNQRSGVPSRFSGSGSGTDFTLTISSLQPEDFATYFCQQSKEVPWTFGQGTKVEIK

SEQ ID NO: 4 - protein, 492 aa, synthetic construct
MDWTWILFLVAAATRVHSEVQLVESGGGLVQPGGSLRLSCSFSGFSLSTSGMGVGWIRQAPGKGLEWVAHIWWDDDKRYNPALKSRFTLSVDRSKNTLYLQMNSLRAEDTATYYCVQINYGNYRFDNWGHGTLVTVSSGGGGSGGGGSGGGGSDIQMTQSPSSLSASVGDRVTISCRASESVDNYGISFLNWFQQKPGKAPKLLIYAASNQRSGVPSRFSGSGSGTDFTLTISSLQPEDFATYFCQQSKEVPWTFGQGTKVEIKTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 5 - protein, 491 aa, synthetic construct
MDWTWILFLVAAATRVHSEVQLVESGGGLVQPGGSLRLSCSFSGFSLSTSGMGVGWIRQAPGKGLEWVAHIWWDDDKRYNPALKSRFTLSVDRSKNTLYLQMNSLRAEDTATYYCVQINYGNYRFDNWGHGTLVTVSSGGGGSGGGGSGGGGSDIQMTQSPSSLSASVGDRVTISCRASESVDNYGISFLNWFQQKPGKAPKLLIYAASNQRSGVPSRFSGSGSGTDFTLTISSLQPEDFATYFCQQSKEVPWTFGQGTKVEIKTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 6 - protein, 533 aa, synthetic construct
MDWTWILFLVAAATRVHSEVQLVESGGGLVQPGGSLRLSCSFSGFSLSTSGMGVGWIRQAPGKGLEWVAHIWWDDDKRYNPALKSRFTLSVDRSKNTLYLQMNSLRAEDTATYYCVQINYGNYRFDNWGHGTLVTVSSGGGGSGGGGSGGGGSDIQMTQSPSSLSASVGDRVTISCRASESVDNYGISFLNWFQQKPGKAPKLLIYAASNQRSGVPSRFSGSGSGTDFTLTISSLQPEDFATYFCQQSKEVPWTFGQGTKVEIKTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2024/0398951 A1
(43) Pub. Date: Dec. 5, 2024

(54) BISPECIFIC T-CELL ENGAGERS TARGETING FSHR AND METHODS OF USE IN CANCER THERAPUTICS

(71) Applicant: The Wistar Institute of Anatomy and Biology, Philadelphia, PA (US)

(72) Inventors: David Weiner, Merion, PA (US); Alfredo Perales-Puchalt, Philadelphia, PA (US); Devivasha Bordoloi, Philadelphia, PA (US); Pratik Bhojnagarwala, Philadelphia, PA (US)

(21) Appl. No.: 18/698,992

(22) PCT Filed: Oct. 6, 2022
(86) PCT No.: PCT/US2022/077665

§ 371 Date: Apr. 5, 2024

Related U.S. Application Data
(60) Provisional application No. 63/377,473, filed on Sep. 28, 2022.
(60) Provisional application No. 63/252,727, filed on Oct. 6, 2021.

(51) Int. Cl. A61K 39/00; A61P 35/00; C07K 14/725; C07K 16/28

(52) U.S. Cl. CPC A61K 39/464429; A61K 39/4611; A61K 39/4631; A61P 35/00; C07K 14/7051; C07K 16/2869; C07K 2317/622

(57) ABSTRACT

Disclosed herein are compositions comprising a recombinant nucleic acid sequences encoding a bispecific anti-FSHR T cell engager as well as FSHR-specific CAR molecules, fragments thereof, variants thereof, combinations thereof, and methods of use thereof.

### CROSS REFERENCE TO RELATED APPLICATIONS

This application claims priority to U.S. Provisional Patent Application No. 63/252,727, filed Oct. 6, 2021 and to U.S. Provisional Patent Application No. 63/377,473, filed Sep. 28, 2022, each of which is hereby incorporated by reference herein in its entirety.

### STATEMENT REGARDING FEDERALLY SPONSORED RESEARCH OR DEVELOPMENT

This invention was made with government support from the U.S. Department of Defense ARMY under Award No. W81XWH-19-1-0189. The government has certain rights in the invention.

### BACKGROUND OF THE INVENTION

Ovarian cancer (OC) represents the deadliest gynecologic malignancy. It stands as the fifth major driver of deaths from cancer among women accounting for the highest number of deaths for cancer of the female reproductive system. As per the American Cancer Society, it is estimated that there were 21,410 women with a new ovarian cancer diagnosis and 13,770 deaths due to OC in 2021 (Kunit et al., 2021, Obstet Gynecol. 2021; 137 (1): 108-21; Key Statistics for Ovarian Cancer. Accessed Nov. 22, 2021. cancer.org). OC is a highly heterogeneous cancer where 90% of tumors are of epithelial origin. The most prevalent subtype of epithelial ovarian cancer (EOC) is high-grade serous cancer constituting around 70-80% of cases, whereas low-grade serous (<5%), endometrioid (10%), clear cell (10%) and mucinous (3%) represent less predominant subtypes (Barnes et al., 2021, Genome Med, 13 (1): 140).

Surgery and chemotherapy are the primary treatments for OC (Yang et al., 2020, Front Immunol. 11: 577869). These approaches are partially successful with many patients developing chemoresistance within a few years after their initial treatment who are next faced with disease recurrence (Yang et al., 2020, Front Immunol. 11: 577869). OC high mortality is also linked to low rates of early detection often due to the lack of subjective symptoms as well as marginally invasive techniques for primary detection. Therefore OC remains a critical need area for novel therapeutic approaches (Banno et al., 2014, Biomed Res Int. 2014: 232817). There is a close interaction between the ovarian tumor cells and the tumor microenvironment, development of treatment approaches which not only target the tumor cells but also can maintain their anti-tumor function in this microenvironment is of particular importance (CSSOCR. Ovarian Cancers: Evolving Paradigms in Research and Care. Washington (DC): National Academies Press (US); 2016). A growing area of study are immune based therapies for OC. Such studies include immune checkpoint inhibitors (ICIs), chimeric antigen receptor (CAR)- and T cell receptor (TCR)-engineered T cells (Yang et al., 2020, Front Immunol. 11:577869). Notably, a prime obstacle in the development of CAR therapies is to find targets with specific expression confined to the surface of tumor cells, but not on healthy tissues (Perales-Puchalt et al., 2017, Clin Cancer Res. 23 (2): 441-500). The follicle-stimulating hormone receptor (FSHR) is one important target reported to have selective expression in ovarian granulosa cells versus low levels of expression in the normal ovarian endothelium. FSHR is expressed in 50-70% of serous ovarian carcinoma cases providing an important potential target for immune therapies (Perales-Puchalt et al., 2017, Clin Cancer Res. 23 (2): 441-500).

Monoclonal antibody therapy has been a game-changer in cancers therapeutics, however, this treatment has several limitations including requirement for repeated administration, more limited stability and cost. A further advance on monoclonal technology is the development of bispecific T cell engagers (BiTE) which combine the specificity of monoclonal antibodies with the cytotoxic potential of T cells. BiTEs have shown promising results in leukemia clinical trials (Viardot et al., 2016, Blood, 127 (11): 1410-6; Goebeler et al., 2016, J Clin Oncol, 34 (10): 1104-11), however, this therapy has a limited applicability because it requires continuous intravenous infusion for 4-8 weeks per cycle (Zhu et al., 2016, Clin Pharmacokinet, 55 (10): 1271-88) and can have limitations for its production. A longer-lived simpler production method for antibody-based products would likely be an important new tool for cancer immunotherapy.

Thus there is need in the art for longer-lived, simpler production, antibody-based products for cancer immunotherapy. The current invention satisfies this need.

### SUMMARY

In one embodiment, the invention relates to a synthetic bispecific immune cell engager (BICE), wherein the synthetic bispecific immune cell engager comprises at least one least one follicle stimulating hormone receptor (FSHR) antigen binding domain, and at least one immune cell engaging domain.

In one embodiment, the immune cell engaging domain targets a T cell, an antigen presenting cell, a natural killer (NK) cell, a neutrophil or a macrophage.

In one embodiment, the immune cell engaging domain targets at least one T cell specific receptor molecule selected from the group consisting of CD3, the T cell receptor (TCR), CD28, CD16, NKG2D, Ox40, 4-1BB, CD2, CD5, CD40, FcgRs, FceRs, FcaRs and CD95. In one embodiment, the immune cell engaging domain targets CD3.

In one embodiment, the BICE comprises an amino acid sequence having at least about 90% identity over an entire length of the amino acid sequence to SEQ ID NO:2. In one embodiment, the BICE comprises a fragment of an amino acid sequence having at least about 90% identity over at least 65% of SEQ ID NO:2. In one embodiment, the BICE comprises an amino acid sequence of SEQ ID NO:2. In one embodiment, the BICE comprises a fragment of an amino acid sequence comprising at least 65% of an amino acid sequence of SEQ ID NO:2.

In one embodiment, the invention relates to a nucleic acid molecule encoding a synthetic bispecific immune cell engager (BICE), wherein the synthetic bispecific immune cell engager comprises at least one least one follicle stimulating hormone receptor (FSHR) antigen binding domain, and at least one immune cell engaging domain. In one embodiment, the nucleic acid molecule an RNA molecule or a DNA molecule. In one embodiment, the nucleic acid molecule comprises a nucleotide sequence having at least about 90% identity over an entire length of the nucleic acid sequence to SEQ ID NO: 1. In one embodiment, the nucleic acid molecule comprises a fragment of a nucleotide sequence having at least about 90% identity over at least 65% of the nucleic acid sequence to SEQ ID NO: 1. In one embodiment, the nucleic acid molecule comprises a nucleotide sequence of SEQ ID NO: 1. In one embodiment, the nucleic acid molecule comprises a fragment of a nucleotide sequence comprising at least 65% of a nucleotide sequence of SEQ ID NO: 1. In one embodiment, the nucleic acid molecule comprises an expression vector.

In one embodiment, the invention relates to a composition comprising a synthetic bispecific immune cell engager (BICE), wherein the synthetic bispecific immune cell engager comprises at least one least one follicle stimulating hormone receptor (FSHR) antigen binding domain, and at least one immune cell engaging domain or a nucleic acid molecule encoding the same.

In one embodiment, the composition further comprises a pharmaceutically acceptable excipient. In one embodiment, the composition further comprises at least one immune checkpoint inhibitor or a nucleic acid molecule encoding the same.

In one embodiment, the immune checkpoint inhibitor is an inhibitor of PD-1, an inhibitor of PD-L-1, an inhibitor of cytotoxic T-lymphocyte antigen-4 (CTLA-4), an inhibitor of mucin-domain containing-3 (TIM-3), or an inhibitor of Lymphocyte Activating 3 (LAG3).

In one embodiment, the composition comprises a lipid nanoparticle comprising a synthetic bispecific immune cell engager (BICE), wherein the synthetic bispecific immune cell engager comprises at least one least one follicle stimulating hormone receptor (FSHR) antigen binding domain, and at least one immune cell engaging domain or a nucleic acid molecule encoding the same.

In one embodiment, the invention relates to a method of preventing or treating a disease or disorder associated with FSHR expression in a subject, the method comprising administering to the subject a synthetic bispecific immune cell engager (BICE), wherein the synthetic bispecific immune cell engager comprises at least one least one follicle stimulating hormone receptor (FSHR) antigen binding domain, and at least one immune cell engaging domain or a nucleic acid molecule encoding the same. In some embodiments, the invention relates to a method of preventing or treating a disease or disorder associated with FSHR expression in a subject, the method comprising administering to the subject a composition comprising a synthetic bispecific immune cell engager (BICE), wherein the synthetic bispecific immune cell engager comprises at least one least one follicle stimulating hormone receptor (FSHR) antigen binding domain, and at least one immune cell engaging domain or a nucleic acid molecule encoding the same. In some embodiments, the composition comprises a combination of synthetic bispecific immune cell engager comprises at least one least one follicle stimulating hormone receptor (FSHR) antigen binding domain, and at least one immune cell engaging domain and at least one immune checkpoint inhibitor or a nucleic acid molecule encoding the same.

In one embodiment, the disease is a benign tumor, cancer or a cancer-associated disease. In one embodiment, the disease is ovarian cancer, breast cancer, prostate cancer, renal cancer, colo-rectal cancer, stomach cancer, lung cancer, testicular cancer, endometrial cancer, or thyroid cancer.

In one embodiment, the invention relates to an FSHR scFv molecule. In one embodiment, the FSHR scFv molecules comprises an amino acid sequence having at least about 90% identity over the entire length of the amino acid sequence of SEQ ID NO:3. In one embodiment, the FSHR scFv molecules comprises a fragment of an amino acid sequence having at least about 90% identity over at least 65% of SEQ ID NO:3. In one embodiment, the FSHR scFv molecules comprises an amino acid sequence of SEQ ID NO: 3. In one embodiment, the FSHR scFv molecules comprises a fragment of an amino acid sequence comprising at least 65% of the amino acid sequence of SEQ ID NO:3.

In one embodiment, the invention relates to a composition comprising an FSHR scFv molecule. In one embodiment, the composition comprises a pharmaceutically acceptable excipient. In one embodiment, the composition comprises at least one immune checkpoint inhibitor. In one embodiment, the immune checkpoint inhibitor an inhibitor of PD-1, an inhibitor of PD-L-1, an inhibitor of cytotoxic T-lymphocyte antigen-4 (CTLA-4), an inhibitor of mucin-domain containing-3 (TIM-3), or an inhibitor of Lymphocyte Activating 3 (LAG3).

In one embodiment, the compostion comprises a lipid nanoparticle comprising an FSHR scFv.

In one embodiment, the composition comprises a CAR molecule comprising an FSHR scFv. In one embodiment, the CAR molecule comprises SEQ ID NO:4, SEQ ID NO: 5 or SEQ ID NO:6.

In one embodiment, the compostion comprises a cell expressing an FSHR scFv or a CAR molecule comprising an FSHR scFv. In one embodiment, the cell is a T cell expressing an FSHR scFv or a CAR molecule comprising an FSHR scFv.

In one embodiment, the invention relates to an FSHR-specific chimeric antigen receptor (CAR) molecule. In one embodiment, the CAR comprises an FSHR specific scFv comprising a nucleotide sequence as set forth in SEQ ID NO:3. In one embodiment, the CAR comprises a sequence of SEQ ID NO:4, SEQ ID NO:5 or SEQ ID NO: 6.

In one embodiment, the invention relates to a composition comprising an FSHR-specific chimeric antigen receptor (CAR) molecule. In one embodiment, the CAR comprises an FSHR specific scFv comprising a nucleotide sequence as set forth in SEQ ID NO: 3. In one embodiment, the CAR comprises a sequence of SEQ ID NO:4, SEQ ID NO:5 or SEQ ID NO:6.

In one embodiment, the composition comprises a cell expressing the FSHR-specific chimeric antigen receptor (CAR) molecule. In one embodiment, the cell is an engineered CAR T cell.

In one embodiment, the invention relates to a method of preventing or treating a disease or disorder associated with FSHR expression in a subject, the method comprising administering to the subject an FSHR scFv antibody fragment, a CAR molecule comprising an FSHR scFv antibody fragment or a composition comprising an FSHR scFv antibody fragment, or a CAR molecule comprising an FSHR scFv antibody fragment. In one embodiment, the method comprises administering an engineered CAR T cell comprising a CAR molecule comprising an FSHR scFv antibody fragment.

In one embodiment, the disease is a benign tumor, cancer or a cancer-associated disease. In one embodiment, the disease is ovarian cancer, breast cancer, prostate cancer, renal cancer, colo-rectal cancer, stomach cancer, lung cancer, testicular cancer, endometrial cancer, or thyroid cancer.

### BRIEF DESCRIPTION OF THE DRAWINGS

FIG. 1 data demonstrating the binding of FSHR bi-specific antibody to K562-FSHR cells.

FIG. 2 depicts data demonstrating that FSHR bi-specific ab kills Ovcar3 cells in the presence of T cells. Effector Cells: T cells; Effector: Target 10:1; Effector cells and treatments were given at 23.8 hr.

FIG. 3 depicts data demonstrating the cytotoxic effect of the FSHRxCD3 bispecific antibody evaluated using xCelligence in OVISE (ovarian clear cell adenocarcinoma) cells.

FIG. 4 depicts data demonstrating the cytotoxic effect of the FSHRxCD3 bispecific antibody evaluated using xCelligence in CaOV3 (High grade ovarian serous adenocarcinoma) cells.

FIG. 5 depicts data demonstrating that in vivo launched FSHRxCD3 bi-specific controls the growth of Ovcar3 tumor cells in NSG mice.

FIG. 6A through FIG. 6F: Generation of anti-human FSHR antibodies. (A) Depiction of FSHR structure. (B) Cloning strategy into pBMN-I-GFP expression vector. (C) Mouse immunization scheme. (D) cAMP response to different doses of FSH hormone of K562 and K562-FSHR. (E) Western blot of phospho-Phospho-p44/42 (Erk 1/2) and p44/42 (Erk1/2) 20 minutes after stimulation of K562 and K562-FSHR cells using FSH. (F) Partially block of cAMP production in K562-FSHR cells by D2AP11 anti-FSHR antibody upon FSH stimulation of FSHR. ANOVA. *** p<0.001, ns: not significant.

FIG. 7A through FIG. 7D: Screening of anti-human FSHR antibodies. (A) Scheme of flow cytometry plots representing the potential outcomes in the screening process with K562 and K562-FSHR. (B) Flow cytometry plot of K562 (GFP-)/K562-FSHR (GFP+) cells stained with sera from mice immunized with human FSHR or empty vector at 1:1000 dilution and anti-mouse IgG APC. (C) Representative of flow cytometric screening output strategy for detection of FSHR binding antibodies from hybridomas as flow plot and fold mean fluorescent intensity of K562-FSHR/K562 (D) Waterfall plot depicting the hybridoma supernatant binding to FSHR measured as fold-MFI K562-FSHR/K562. After the first round of screening, the top 20 clones (left of the red bar) were selected for further study.

FIG. 8A through FIG. 8B: Binding potency of D2AP11 and commercial mouse anti-human FSHR antibodies. (A) Binding of D2AP11 anti-FSHR Ab and 4 different commercial Abs (Commercial Ab #1, 2, 3&4) in FSHR overexpressing K562 cells analyzed by flow cytometry. Only secondary Ab control and Irrelevant Ab controls are shown in the left, where no binding to K562 and K562-FSHR cells were observed. The binding with different anti-FSHR antibodies were evaluated at different concentrations: 2500 ng/ml, 1250 ng/ml, 625 ng/ml, 312.5 ng/ml, 156.25 ng/ml and 78.125 ng/ml. (B) Dose dependent binding of D2AP11 anti-FSHR Ab to K562-FSHR cells analyzed by flow cytometry. D2AP11 binding was observed at concentrations as low as 9.77 ng/ml, indicating its potency.

FIG. 9A through FIG. 9C: Binding of D2AP11 to healthy and ovarian cancer tissues. (A) Representative images showing the binding of D2AP11 anti-FSHR antibody to healthy tissues of different human organs (pancreas, lung, heart, small intestine, colon, uterus, ovary and fallopian tube endothelium), analyzed by immunohistochemistry/IHC. (B) Representative images showing the binding of D2AP11 anti-FSHR antibody to ovarian cancer tissues of different pathological conditions (High and low-grade serous carcinoma, mucinous adenocarcinoma, clear cell carcinoma, dysgerminoma, endodermal sinus carcinoma, and metastatic adenocarcinoma), analyzed by IHC staining of ovarian cancer TMA (US Biomax). The tissues (A & B) were viewed and imaged using Nikon NIS-Element Imaging system (20×, scale: 500 μm). Images were subjected to post acquisition adjustments to optimize brightness, contrast, and image visibility. (C) Comparison of normalized RNA expression of FSHR and ERBB2/Her2 on 55 human tissue types, based on ‘The Human Protein Atlas’ data.

FIG. 10A through FIG. 10B: Differential expression of FSHR and ERBB2/Her2 in 55 tissue types. (A) Differential expression (RNA) of FSHR in 55 different human tissue types, as analyzed using ‘The Human Protein Atlas’ database. (B) Differential expression (RNA) of ERBB2/Her2 in different human tissues, as analyzed using ‘The Human Protein Atlas’ database (proteinatlas.org); nTPM: normalized expression level.

FIG. 11A through FIG. 11D: D2AP11 binds human and murine FSHR. (A) Flow cytometry plot of CaOV3, OVCAR3 and TOV-21G stained with D2AP11 or no primary antibody followed secondary APC labelled antibody. (B) Flow cytometry plot of TOV-21G parental or after CRISPR of FSHR stained with D2AP11. (C) Flow cytometry plot of K562, K562-FSHR and K562-LHCGR 21G stained with D2AP11 or no primary antibody followed by secondary APC labelled antibody. (D) Flow cytometry plot of A20 (GFP-)/A20-Fhsr (GFP+) and ID8-Defb29 Vegf-a vs. ID8-Defb29 Vegf-a-Fshr cells stained with D2AP11 (both cell lines were transfected with murine FSHR).

FIG. 12A through FIG. 12G: D2AP11 binds to FSHR in immunohistochemistry and immunocytochemistry and induces antibody-dependent cell mediated cytotoxicity. (A) Immunohistochemistry images from frozen sections of tumors derived from K562, K562-FSHR, OVCAR3 and TOV-21G cell lines stained with D2AP11. 40×, Scale bar 50 μm. (B) Immunofluorescence images of 293T cells transfected with human FSHR and stained with either mouse anti-human FSHR or D2AP11 antibodies followed by secondary anti-mouse IgG. (C) Immunofluorescence images of untransfected 293T cells and stained with D2AP11 antibodies followed by secondary anti-mouse IgG. B-D: Scale bar 10 μm. (D) Absorbance values of isotype ELISA performed on D2AP11 antibody. (E) Cytotoxicity mediated by ADCC of D2AP11 or irrelevant mouse IgG2a (C1.18.4) against K562-FSHR. (F) Cytotoxicity mediated by ADCC of D2AP11 or irrelevant mouse IgG2a (C1.18.4) against K562. (G) Cytotoxicity mediated by ADCC of D2AP11 or irrelevant mouse IgG2a (C1.18.4) against OVCAR3 cells. t-test, ANOVA. *** p<0.001, ns not significant.

FIG. 13A through FIG. 13H: Generation, expression, and antitumor activity of FSHR TCE. (A) Cartoon of TCE engaging FSHR and the T cell receptor (TCR). GS, glycine-serine; VH, heavy chain variable region; VL, light chain variable region. (B) Schematic of DNA construct encoding D2AP11-TCE. (C) Western blot of in vitro expression of D2AP11-TCE or pVax1 empty vector after transfection in Expi293F cells. (D) The binding specificity of D2AP11-TCE was verified using K562 cells, which lack natural expression of FSHR. In FSHR-non expressing K562 cells, no binding of D2AP11-TCE was overserved. (E) Binding of D2AP11-TCE to FSHR overexpressing K562 cells. (F) Binding of D2AP11-TCE to FSHR shown using additional FSHR expressing cell; CaOV3. Shift in the peak in D2AP11-TCE compared to pVax1 and secondary Ab alone indicates its binding to FSHR. (G) Binding of D2AP11-TCE to FSHR shown using OVCAR3 cells transduced with FSHR encoding pBMN-I-GFP plasmid for overexpression of FSHR. There is a remarkable shift in peak in FSHR overexpressing OVCAR3 cells compared to empty vector and secondary antibody alone control. (H) Flow staining of primary human T cells with D2AP11-TCE and empty vector control, shift in peak denotes the binding of D2AP11-TCE to human T cells.

FIG. 14A through FIG. 14P: D2AP11-TCE induce specific killing of target ovarian cancer cells. Assessing the cytotoxic effect of D2AP11-TCE in FSHR negative (A&B) HEK 293 cells, (C&D) AGS gastric adenocarcinoma cells and (E) WM3743 human melanoma cells as well as in target human ovarian cancer cells (F&G) OVISE cells, (H&I) CaOV3 cells, (J) OVCAR4 cells, (K&L) OVCAR3-FSHR cells, (M) PEO-4 cells, and (N) Kuramochi-FSHR cells, dose dependent killing of (O) OVISE-FSHR and (P) OVCAR3 cells in the presence of D2AP11-TCE and Human PBMCs. In vitro cytotoxicity was measured based on impedance using xCELLigence real time cell analyzer equipment (RTCA), Agilent Technologies, USA. The electrical conductivity is converted into the unitless cell index (CI) parameter by the xCELLigence device in every 15 minutes and images were captured at the interval of 1 hour. The data generated are normalized as per the time point when the effector (E) cells (PBMCs), and D2AP11-TCE were added to the target (T) cells; E: T is 5:1 (A&B, F&G, M, N) and 10:1 (C-E, H-L, O&P). The data were analyzed using RTCA/RTCA Pro Software. No non-specific killing was obtained in HEK293T, 773 AGS and WM3743 cells, whereas potent killing was observed in OVISE, CaOV3, OVCAR3-FSHR, PEO-4 and Kuramochi-FSHR target OC cells. Arrow indicates the time point at which antibodies and effector cells were added to the target cells. Images shown display killing, 2-3 days after the addition of effector cells and antibodies.

FIG. 15A through FIG. 15C: FSHR targeted bispecific T cell engagers induced ovarian cancer cytotoxicity in the presence of purified T cells. In vitro cytotoxicity resulting from co-culture of T cells with (A) OVCAR3-FSHR cells and (B) OVCAR4 cells in the presence of D2AP11-TCE. The real time in vitro cytotoxicity analysis was done by xCELLigence; E:T=5:1/10:1 (C) Images showing the cytotoxic effect of D2AP11-TCE on OVCAR4 cells in the presence of human T cells, 3 days after the addition of effector cells and antibodies. No Ab (Only Effector+Target) served as control. Arrow indicates the time at which the effector cells and TCE were added.

FIG. 16A through FIG. 16C: Dose dependent killing of OVISE-FSHR cells by D2AP11-TCE. Dose dependent killing observed in OVISE-FSHR cells by D2AP11-TCE in the presence of (A) Human PBMCs and (B) Human T cells. (C) Dose dependent killing of OVCAR3 cells by D2AP11-TCE in the presence of Human PBMCs. The real time in vitro cytotoxicity analysis was done by xCELLigence; E:T=10:1. D2AP11-TCE was added at the indicated concentrations ranging from 500 ng/ml to 7.81 ng/ml.

FIG. 17A: Irrelevant TCE did induce killing in FSHR overexpressing ovarian cancer cells. IL13Rα2-TCE (an irrelevant/non FSHR targeting TCE) did not cause cytotoxicity to FSHR overexpressing OVCAR3 cells in the presence of human PBMCs. Arrow indicates the time at which the effector cells and TCE were added. Red line indicates no Ab control (Only Effector+Target cells); E:T=5:1.

FIG. 18A through FIG. 18D: Comparative cell killing analysis of D2AP11 and D2AP11-TCE. (A) Killing of OVCAR4 cells by D2AP11 anti-FSHR antibody at the indicated concentrations in presence of human PBMCs. (B) Killing of OVCAR4 cells by D2AP11-TCE at the indicated concentrations in presence of human PBMCs. Arrow indicates the time at which the effector cells and D2AP11/D2AP11-TCE was added. (C) D2AP11 anti-FSHR ab exhibited dose dependent killing of OVCAR4 cells with EC50 value of 30.3 μg/ml, in presence of human PBMCs (D) D2AP11-TCE displayed dose dependent killing of OVCAR4 cells with EC50 value of 11.3 ng/ml, in the presence of human PBMCs, indicating ˜1000-fold higher potency compared to the anti-FSHR antibody. The real time in vitro cytotoxicity analysis was done by xCELLigence; E:T=10:1. EC50 values were calculated using RTCA Pro software.

FIG. 19A through FIG. 19F: Cytokine secretion profile and in vivo activity of D2AP11-TCE. (A) Secretion profile of cytokines; IFN-gamma, sFas, granzymes A&B and perforin in the presence of D2AP11-TCE upon co culturing of OVCAR3-FSHR and human PBMCs; E:T=10:1. The supernatants analyzed for cytokine secretion profile were collected 48 hours after the addition of effectors cells and TCE to target OVCAR3-FSHR cells. PBMCs from 3 different donors were used; two-tailed unpaired Student's t test; * p<0.05, ** p<0.01, *** p<0.001. (B) Schematic of tumor study to evaluate the effect of D2AP11-TCE on tumor progression in K562/K562-FSHR challenged NSG mice model. (C) Average growth curve of K562 tumors grafted into NSG mice treated with D2AP11-TCE or empty vector (n=5 mice per group). (D) Average growth curve of K562-FSHR tumors grafted into NSG mice treated with D2AP11-TCE or empty vector (n=5 mice per group). (E) Schematic of tumor study to evaluate the effect of D2AP11-TCE on tumor progression in OVCAR3-FSHR challenged NSG mice model. (F) Average growth curve of 36 OVCAR3-FSHR tumors grafted into NSG mice treated with D2AP11-TCE or empty vector (n=10 mice per group). Two-way ANOVA; * p<0.05, ** p<0.01, *** p<0.001.

FIG. 20 shows that D2AP11-BTE synergistically enhances the killing by Nivolumab (anti-PD1 antibody) in ovarian cancer cells. Arrows indicate the time at which effector cells and treatment was given.

FIG. 21 shows that D2AP11-BTE synergistically enhances the killing by Pembrolizumab (anti-PD1 antibody) in ovarian cancer cells. Arrows indicate the time at which effector cells and treatment was given.

FIG. 22 shows that D2AP11-BTE synergistically enhances the killing by anti-CTLA4 antibodies in ovarian cancer cells. Arrows indicate the time at which effector cells and treatment was given.

FIG. 23 shows a diagram depicting CAR T cells that have been developed based on the 9h11 (D2AP11) antibody sequence.

### DETAILED DESCRIPTION

The present invention relates to compositions comprising FSHR-specific binding molecules including a bispecific immune cell engaging antibody (BICE), a bispecific T cell engaging (BiTE) antibody, an scFv antibody fragment and CAR molelcules, fragments thereof, variants thereof, combinations thereof and nucleic acid molecules encoding the same.

In one embodiment, the BICE or BiTE comprisies at least one antigen binding domain, and at least one immune cell engaging domain. In one embodiment, the immune cell engaging domain is specific for an antigen expressed on the surface of an immune cell. Immune cells include, but are not limited to, T cells, antigen presenting cells, NK cells, neutrophils and macrophages.

In various embodiments, the immune cell engaging domain comprises a nucleotide sequence encoding an antibody, a fragment thereof, or a variant thereof specific for binding to a immune cell specific receptor molecule. In one embodiment, the immune cell specific receptor molecule is a T cell surface antigen. In one embodiment, the T cell specific receptor molecule is one of CD3, TCR, CD28, CD16, NKG2D, Ox40, 4-1BB, CD2, CD5, CD40, FcgRs, FceRs, FcaRs and CD95.

In various embodiments, the antigen binding domain comprises a nucleotide sequence encoding an antibody, a fragment thereof, or a variant thereof specific for binding to an antigen. In one embodiment, the antibody or fragment thereof is a DNA encoded monoclonal antibody (DMAb) or a fragment or variant thereof. In one embodiment, the antibody or fragment thereof is an mRNA encoded monoclonal antibody or a fragment or variant thereof.

In one embodiment, the antigen binding domain of the BICE or BiTE is specific for binding a target antigen, and recruiting a T cell to the target antigen. In one embodiment, the target antigen is a tumor antigen. In one embodiment, the antigen follicle stimulating hormone receptor (FSHR). Therefore, in one embodiment, the invention provides compositions comprising one or more BICE or BiTE and methods for use in treating or preventing cancer or a disease or disorder associated with cancer in a subject.

In one embodiment, the invention relates to CAR T cells expressing CAR molecules comprising an antigen binding domain specific for binding to FSHR. In some emboidments, the FSHR binding domain comprises an scFv antibody fragment specific for binding to FSHR. In some embodiments, the scFv comprises a sequence as set forth in SEQ ID NO: 3. In some embodiment, the CAR molecules of the invention provides for both co-stimulation by CD28 and/or 4-1BB domains, and activation, by a CD3ζ signaling domain. In the embodiments disclosed herein, the CAR comprises a sequence as set forth in SEQ ID NO: 4, SEQ ID NO:5 or SEQ ID NO:6 or a fragment or variant thereof.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a.” “and” and “the” include plural references unless the context clearly dictates otherwise. The present disclosure also contemplates other embodiments “comprising.” “consisting of” and “consisting essentially of,” the embodiments or elements presented herein, whether explicitly set forth or not.

“Antibody” may mean an antibody of classes IgG, IgM, IgA, IgD or IgE, or fragments, fragments or derivatives thereof, including Fab, F(ab′)2, Fd, and single chain antibodies, and derivatives thereof. The antibody may be an antibody isolated from the serum sample of mammal, a polyclonal antibody, affinity purified antibody, or mixtures thereof which exhibits sufficient binding specificity to a desired epitope or a sequence derived therefrom.

“Antibody fragment” or “fragment of an antibody” as used interchangeably herein refers to a portion of an intact antibody comprising the antigen-binding site or variable region. The portion does not include the constant heavy chain domains (i.e. CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)2 fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

“Antigen” refers to proteins that have the ability to generate an immune response in a host. An antigen may be recognized and bound by an antibody. An antigen may originate from within the body or from the external environment.

“Coding sequence” or “encoding nucleic acid” as used herein may mean refers to the nucleic acid (RNA or DNA molecule) that comprise a nucleotide sequence which encodes an antibody as set forth herein. The coding sequence may further include initiation and termination signals operably linked to regulatory elements including a promoter and polyadenylation signal capable of directing expression in the cells of an individual or mammal to whom the nucleic acid is administered. The coding sequence may further include sequences that encode signal peptides.

“Complement” or “complementary” as used herein may mean a nucleic acid may mean Watson-Crick (e.g., A-T/U and C-G) or Hoogsteen base pairing between nucleotides or nucleotide analogs of nucleic acid molecules.

“Endogenous antibody” as used herein may refer to an antibody that is generated in a subject that is administered an effective dose of an antigen for induction of a humoral immune response.

“Feedback mechanism” as used herein may refer to a process performed by either software or hardware (or firmware), which process receives and compares the impedance of the desired tissue (before, during, and/or after the delivery of pulse of energy) with a present value, preferably current, and adjusts the pulse of energy delivered to achieve the preset value. A feedback mechanism may be performed by an analog closed loop circuit.

“Fragment” may mean a polypeptide fragment of an antibody that is function, i.e., can bind to desired target and have the same intended effect as a full length antibody. A fragment of an antibody may be 100% identical to the full length except missing at least one amino acid from the N and/or C terminal, in each case with or without signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length antibody, excluding any heterologous signal peptide added. The fragment may comprise a fragment of a polypeptide that is 95% or more, 96% or more, 97% or more, 98% or more or 99% or more identical to the antibody and additionally comprise an N terminal methionine or heterologous signal peptide which is not included when calculating percent identity. Fragments may further comprise an N terminal methionine and/or a signal peptide such as an immunoglobulin signal peptide, for example an IgE or IgG signal peptide. The N terminal methionine and/or signal peptide may be linked to a fragment of an antibody.

A fragment of a nucleic acid sequence that encodes an antibody may be 100% identical to the full length except missing at least one nucleotide from the 5′ and/or 3′ end, in each case with or without sequences encoding signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length coding sequence, excluding any heterologous signal peptide added. The fragment may comprise a fragment that encode a polypeptide that is 95% or more, 96% or more, 97% or more, 98% or more or 99% or more identical to the antibody and additionally optionally comprise sequence encoding an N terminal methionine or heterologous signal peptide which is not included when calculating percent identity. Fragments may further comprise coding sequences for an N terminal methionine and/or a signal peptide such as an immunoglobulin signal peptide, for example an IgE or IgG signal peptide. The coding sequence encoding the N terminal methionine and/or signal peptide may be linked to a fragment of coding sequence.

“Genetic construct” as used herein refers to the DNA or RNA molecules that comprise a nucleotide sequence which encodes a protein, such as an antibody. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Identical” or “identity” as used herein in the context of two or more nucleic acids or polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Impedance” as used herein may be used when discussing the feedback mechanism and can be converted to a current value according to Ohm's law; thus enabling comparisons with the preset current.

“Immune response” as used herein may mean the activation of a host's immune system, e.g., that of a mammal, in response to the introduction of one or more nucleic acids and/or peptides. The immune response can be in the form of a cellular or humoral response, or both.

“Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

“Operably linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

A “peptide,” “protein,” or “polypeptide” as used herein can mean a linked sequence of amino acids and can be natural, synthetic, or a modification or combination of natural and synthetic.

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40) early promoter or SV 40 late promoter and the CMV IE promoter.

“Signal peptide” and “leader sequence” are used interchangeably herein and refer to an amino acid sequence that can be linked at the amino terminus of a protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

“Stringent hybridization conditions” as used herein may mean conditions under which a first nucleic acid sequence (e.g., probe) will hybridize to a second nucleic acid sequence (e.g., target), such as in a complex mixture of nucleic acids. Stringent conditions are sequence dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10)-50 nucleotides) and at least about 60° C. for long probes (e.g., greater than about 50) nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc) and a human). In some embodiments, the subject may be a human or a non-human. The subject or patient may be undergoing other forms of treatment.

“Substantially complementary” as used herein may mean that a first sequence is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under stringent hybridization conditions.

“Substantially identical” as used herein may mean that a first and second sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides or amino acids, or with respect to nucleic acids, if the first sequence is substantially complementary to the complement of the second sequence.

“Synthetic antibody” as used herein refers to an antibody that is encoded by the recombinant nucleic acid sequence described herein and is generated in a subject.

“Treatment” or “treating,” as used herein can mean protecting of a subject from a disease through means of preventing, suppressing, repressing, or completely eliminating the disease. Preventing the disease involves administering an antibody of the present invention to a subject prior to onset of the disease. Suppressing the disease involves administering a antibody of the present invention to a subject after induction of the disease but before its clinical appearance. Repressing the disease involves administering an antibody of the present invention to a subject after clinical appearance of the disease.

“Variant” used herein with respect to a nucleic acid may mean (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

“Vector” as used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### Compositions

In one embodiment, the present invention relates to compositions comprising a bispecific immune cell engager (or BICE) or bispecific T cell engager (or BiTE), a fragment thereof, a variant thereof, or a combination thereof. The compositions, when administered to a subject in need thereof, can result in the generation of a synthetic bispecific immune cell engager in the subject. In some embodiments, the BICE or BiTE of the invention is encoded by a nucleic acid molecue (e.g., DNA or mRNA). Therefore, in some embodiments, the invention relates to nucleic acid molecules encoding a BICE or BiTE.

In one embodiment, the BICE or BiTE comprisies at least one antigen binding domain, and at least one immune cell engaging domain. In one embodiment, the immune cell engaging domain is specific for an antigen expressed on the surface of an immune cell. Immune cells include, but are not limited to, T cells, antigen presenting cells, NK cells, neutrophils and macrophages.

In various embodiments, the immune cell engaging domain comprises a nucleotide sequence encoding an antibody, a fragment thereof, or a variant thereof specific for binding to a immune cell specific receptor molecule. In one embodiment, the immune cell specific receptor molecule is a T cell surface antigen. In one embodiment, the T cell specific receptor molecule is one of CD3, TCR, CD28, CD16, NKG2D, Ox40, 4-1BB, CD2, CD5, CD40, FcgRs, FceRs, FcaRs and CD95.

In some embodiments, the invention provides chimeric antigen receptor (CAR) molecules comprising an antigen binding domain and an immune cell activation domain. In some embodiments, the immune cell activation domain comprises at least one intracellular domain from a co-stimulatory molecules, for example, CD28, 4-1BB, ICOS, OX40, and the like.

In various embodiments, the antigen binding domain comprises an antibody, a fragment thereof (e.g., an scFv fragment), or a variant thereof specific for binding to an antigen. In one embodiment, the antigen is a tumor antigen. In one embodiment, the antigen is follicle stimulating hormone receptor (FSHR).

In one embodiment, the FSHR-BiTE comprises the amino acid sequence of SEQ ID NO:2 or a fragment or variant thereof.

In various embodiments, the antigen binding domain comprises an scFv specific for FSHR. In some embodiments, the scFv molelcule comprises an amino acid sequence as set forth in SEQ ID NO:3.

In certain embodiments, the composition can treat, prevent, and or/protect against a disease or disorder associated with FSHR expression. In certain embodiments, the composition can treat, prevent, and or/protect against cancer associated with FSHR expression.

The synthetic antibody (e.g., scFv antibody fragment, BICE or BiTE) or the CAR molecule of the invention, can treat, prevent, and/or protect against disease in the subject administered the composition. The synthetic antibody (e.g., scFv antibody fragment, BICE or BiTE) or the CAR molecule of the invention can promote survival of the disease in the subject administered the composition. The synthetic antibody (e.g., scFv antibody fragment, BICE or BiTE) or the CAR molecule can provide at least about 50%, 55%, 60%, 65%, 70%, 75%, 80%, 85%, 90%, 95%, or 100% survival of the disease in the subject administered the composition. In other embodiments, the synthetic antibody (e.g., scFv antibody fragment, BICE or BiTE) or the CAR molecule can provide at least about 65%, 66%, 67%, 68%, 69%, 70%, 71%, 72%, 73%, 74%, 75%, 76%, 77%, 78%, 79%, or 80% survival of the disease in the subject administered the composition.

The composition can result in the generation of the synthetic antibody (e.g., scFv antibody fragment, BICE or BiTE) or the CAR molecule in the subject within at least about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 20 hours, 25 hours, 30 hours, 35 hours, 40 hours, 45 hours, 50 hours, or 60 hours of administration of the composition to the subject. The composition can result in generation of the synthetic antibody (e.g., scFv antibody fragment, BICE or BiTE) in the subject within at least about 1 day, 2 days, 3 days, 4 days, 5 days, 6 days, 7 days, 8 days, 9 days, or 10 days of administration of the composition to the subject. The composition can result in generation of the synthetic antibody (e.g., scFv antibody fragment, BICE or BiTE) or the CAR molecule in the subject within about 1 hour to about 6 days, about 1 hour to about 5 days, about 1 hour to about 4 days, about 1 hour to about 3 days, about 1 hour to about 2 days, about 1 hour to about 1 day, about 1 hour to about 72 hours, about 1 hour to about 60 hours, about 1 hour to about 48 hours, about 1 hour to about 36 hours, about 1 hour to about 24 hours, about 1 hour to about 12 hours, or about 1 hour to about 6 hours of administration of the composition to the subject.

The composition, when administered to the subject in need thereof, can result in the generation of the synthetic antibody (e.g., scFv antibody fragment, BICE or BiTE) or the CAR molecule in the subject more quickly than the generation of an endogenous antibody in a subject who is administered an antigen to induce a humoral immune response. The composition can result in the generation of the synthetic antibody (e.g., scFv antibody fragment, BICE or BiTE) or the CAR molecule at least about 1 day, 2 days, 3 days, 4 days, 5 days, 6 days, 7 days, 8 days, 9 days, or 10 days before the generation of the endogenous antibody in the subject who was administered an antigen to induce a humoral immune response.

The composition of the present invention can have features required of effective compositions such as being safe so that the composition does not cause illness or death; being protective against illness; and providing ease of administration, few side effects, biological stability and low cost per dose.

### Antibody

In some embodiments, the invention relates to an antibody, a fragment thereof, a variant thereof, or a combination thereof. The antibody can bind or react with an antigen, which is described in more detail below. In some emboidments the fragment is an scFv fragment. In some embodiments, the antibody is a bispecific T cell engagers (BiTE), a fragment thereof, or a variant thereof.

In some embodiments, the antibody may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region. Proceeding from the N-terminus of a heavy or light chain, these regions are denoted as “CDR1,” “CDR2,” and “CDR3,” respectively. An antigen-binding site, therefore, may include six CDRs, comprising the CDR set from each of a heavy and a light chain V region.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)2 fragment, which comprises both antigen-binding sites. Accordingly, the antibody can be the Fab or F(ab′)2. The Fab can include the heavy chain polypeptide and the light chain polypeptide. The heavy chain polypeptide of the Fab can include the VH region and the CH1 region. The light chain of the Fab can include the VL region and CL region.

The antibody can be an immunoglobulin (Ig). The Ig can be, for example, IgA, IgM, IgD, IgE, and IgG. The immunoglobulin can include the heavy chain polypeptide and the light chain polypeptide. The heavy chain polypeptide of the immunoglobulin can include a VH region, a CH1 region, a hinge region, a CH2 region, and a CH3 region. The light chain polypeptide of the immunoglobulin can include a VL region and CL region.

The antibody can be a polyclonal or monoclonal antibody. The antibody can be a chimeric antibody, a single chain antibody, an affinity matured antibody, a human antibody, a humanized antibody, or a fully human antibody. The humanized antibody can be an antibody from a non-human species that binds the desired antigen having one or more complementarity determining regions (CDRs) from the non-human species and framework regions from a human immunoglobulin molecule.

The antibody can be a bispecific antibody as described below in more detail. The antibody can be a bifunctional antibody as also described below in more detail.

The antibody may have a half-life within the subject. In some embodiments, the antibody may be modified to extend or shorten its half-life within the subject. Such modifications are described below in more detail.

### Heavy Chain Polypeptide

The binding molecule of the invention can include a heavy chain polypeptide, a fragment thereof, a variant thereof, or a combination thereof. The heavy chain polypeptide can include a variable heavy chain (VH) region and/or at least one constant heavy chain (CH) region. The at least one constant heavy chain region can include a constant heavy chain region 1 (CH1), a constant heavy chain region 2 (CH2), and a constant heavy chain region 3 (CH3), and/or a hinge region.

In some embodiments, the heavy chain polypeptide can include a VH region and a CH1 region. In other embodiments, the heavy chain polypeptide can include a VH region, a CH1 region, a hinge region, a CH2 region, and a CH3 region.

The heavy chain polypeptide can include a complementarity determining region (“CDR”) set. The CDR set can contain three hypervariable regions of the VH region. Proceeding from N-terminus of the heavy chain polypeptide, these CDRs are denoted “CDR1,” “CDR2,” and “CDR3,” respectively. CDR1, CDR2, and CDR3 of the heavy chain polypeptide can contribute to binding or recognition of the antigen.

### Light Chain Polypeptide

The binding molecule of the invention can include a light chain polypeptide, a fragment thereof, a variant thereof, or a combination thereof. The light chain polypeptide can include a variable light chain (VL) region and/or a constant light chain (CL) region.

The light chain polypeptide can include a complementarity determining region (“CDR”) set. The CDR set can contain three hypervariable regions of the VL region. Proceeding from N-terminus of the light chain polypeptide, these CDRs are denoted “CDR1,” “CDR2,” and “CDR3,” respectively. CDR1, CDR2, and CDR3 of the light chain polypeptide can contribute to binding or recognition of the antigen.

### Linker Sequence

The binding molecule of the invention of the invention can include one or more linker sequences. The linker sequence can spatially separate or link the one or more components described herein. In other embodiments, the linker sequence can comprise an amino acid sequence that spatially separates or links two or more polypeptides. In one embodiment, the linker sequence is a G4S linker sequence.

### Leader Sequence

The binding molecule of the invention of the invention can include one or more leader sequences. In one embodiment, the leader sequence is a signal peptide. The signal peptide can be an immunoglobulin (Ig) signal peptide, for example, but not limited to, an IgG signal peptide and a IgE signal peptide.

### ScFv Antibody

In one embodiment, the binding molecule of the invention comprises a scFv antibody fragment. In one embodiment, scFv relates to a Fab fragment without the CH1 and CL regions. Thus, in one embodiment, the scFv relates to a Fab fragment comprising the VH and VL. In one embodiment, the scFv comprises a linker between VH and VL. In one embodiment, the scFv relates to a Fab fragment comprising the VL and VH. In one embodiment, the scFv comprises a linker between VL and VH. In one embodiment, the scFv fragment is an ScFv-Fc. In one embodiment, the scFv-Fc fragment comprises the VH, VL and the CH2 and CH3 regions. In one embodiment, the scFv-Fc fragment comprises a linker between VH and VL. In one embodiment, the ScFv fragment of the invention has modified expression, stability, half-life, antigen binding, heavy chain-light chain pairing, tissue penetration or a combination thereof as compared to a parental antibody.

In one embodiment, the scFv of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher expression than the parental antibody.

In one embodiment, the scFv fragment of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher antigen binding than the parental antibody.

In one embodiment, the scFv fragment of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold longer half-life than the parental antibody.

In one embodiment, the scFv fragment of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher stability than the parental antibody.

In one embodiment, the scFv fragment of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold greater tissue penetration than the parental antibody.

In one embodiment, the scFv fragment of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold greater heavy chain-light chain pairing than the parental antibody.

### Bispecific T Cell Engager

As described above, the binding molecule of the invention can be a bispecific T cell engager (BiTE), a fragment thereof, a variant thereof, or a combination thereof. The antigen targeting domain of the BiTE can bind or react with the antigen, which is described in more detail below.

The antigen targeting domain of the BiTE may comprise an antibody, a fragment thereof, a variant thereof, or a combination thereof. The antigen targeting domain of the BiTE may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region. Proceeding from the N-terminus of a heavy or light chain, these regions are denoted as “CDR1,” “CDR2,” and “CDR3,” respectively. An antigen-binding domain, therefore, may include six CDRs, comprising the CDR set from each of a heavy and a light chain V region.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)₂ fragment, which comprises both antigen-binding sites. Accordingly, the antigen targeting domain of the BiTE can be the Fab or F(ab′)₂. The Fab can include the heavy chain polypeptide and the light chain polypeptide. The heavy chain polypeptide of the Fab can include the VH region and the CH1 region. The light chain of the Fab can include the VL region and CL region.

The antigen targeting domain of the BiTE can be an immunoglobulin (Ig). The Ig can be, for example, IgA, IgM, IgD, IgE, and IgG. The immunoglobulin can include the heavy chain polypeptide and the light chain polypeptide. The heavy chain polypeptide of the immunoglobulin can include a VH region, a CH1 region, a hinge region, a CH2 region, and a CH3 region. The light chain polypeptide of the immunoglobulin can include a VL region and CL region.

The antigen targeting domain of the BiTE can be a polyclonal or monoclonal antibody. The antibody can be a chimeric antibody, a single chain antibody, an affinity matured antibody, a human antibody, a humanized antibody, or a fully human antibody. The humanized antibody can be an antibody from a non-human species that binds the desired antigen having one or more complementarity determining regions (CDRs) from the non-human species and framework regions from a human immunoglobulin molecule.

In some embodiments, the invention includes nucleic acid molecules (e.g., DNA or mRNA) encoding the BiTE.

In one embodiment, at least one of the antigen binding domain and the immune cell engaging domain of the BiTE of the invention is an scFv antibody fragment as described in detail above.

### Bispecific Antibody

The bispecific T cell engager of the invention can be a bispecific antibody, a fragment thereof, a variant thereof, or a combination thereof. The bispecific antibody can bind or react with two antigens, for example, two of the antigens described below in more detail. The bispecific antibody can be comprised of fragments of two of the antibodies described herein, thereby allowing the bispecific antibody to bind or react with two desired target molecules, which may include the antigen, which is described below in more detail, a ligand, including a ligand for a receptor, a receptor, including a ligand-binding site on the receptor, a ligand-receptor complex, and a marker.

The invention provides novel bispecific antibodies comprising a first antigen-binding site that specifically binds to a first target and a second antigen-binding site that specifically binds to a second target, with particularly advantageous properties such as producibility, stability, binding affinity, biological activity, specific targeting of certain T cells, targeting efficiency and reduced toxicity. In some instances, there are bispecific antibodies, wherein the bispecific antibody binds to the first target with high affinity and to the second target with low affinity. In other instances, there are bispecific antibodies, wherein the bispecific antibody binds to the first target with low affinity and to the second target with high affinity. In other instances, there are bispecific antibodies, wherein the bispecific antibody binds to the first target with a desired affinity and to the second target with a desired affinity.

In one embodiment, the bispecific antibody is a bivalent antibody comprising a) a first light chain and a first heavy chain of an antibody specifically binding to a first antigen, and b) a second light chain and a second heavy chain of an antibody specifically binding to a second antigen.

A bispecific antibody molecule according to the invention may have two binding sites of any desired specificity. In some embodiments, one of the binding sites is capable of an tumor antigen. In some embodiments, the binding site included in the Fab fragment is a binding site specific for a tumor antigen. In some embodiments, the binding site included in the single chain Fv fragment is a binding site specific for a FSHR tumor antigen.

In some embodiments, one of the binding sites of an antibody molecule according to the invention is able to bind a T-cell specific receptor molecule and/or a natural killer cell (NK cell) specific receptor molecule. A T-cell specific receptor is the so called “T-cell receptor” (TCRs), which allows a T cell to bind to and, if additional signals are present, to be activated by and respond to an epitope/antigen presented by another cell called the antigen-presenting cell or APC. The T cell receptor is known to resemble a Fab fragment of a naturally occurring immunoglobulin. It is generally monovalent, encompassing.alpha.- and .beta-chains, in some embodiments, it encompasses.gamma-chains and .delta-chains (supra). Accordingly, in some embodiments, the TCR is TCR (alpha/beta) and in some embodiments, it is TCR (gamma/delta). The T cell receptor forms a complex with the CD3 T-Cell co-receptor. CD3 is a protein complex and is composed of four distinct chains. In mammals, the complex contains a CD3γ chain, a CD36 chain, and two CD3E chains. These chains associate with a molecule known as the T cell receptor (TCR) and the ζ-chain to generate an activation signal in T lymphocytes. Hence, in some embodiments, a T-cell specific receptor is the CD3 T-Cell co-receptor. In some embodiments, a T-cell specific receptor is CD28, a protein that is also expressed on T cells. CD28 can provide co-stimulatory signals, which are required for T cell activation. CD28 plays important roles in T-cell proliferation and survival, cytokine production, and T-helper type-2 development. Yet a further example of a T-cell specific receptor is CD134, also termed Ox40. CD134/OX40 is being expressed after 24 to 72 hours following activation and can be taken to define a secondary costimulatory molecule. Another example of a T-cell receptor is 4-1 BB capable of binding to 4-1 BB-Ligand on antigen presenting cells (APCs), whereby a costimulatory signal for the T cell is generated. Another example of a receptor predominantly found on T-cells is CD5, which is also found on B cells at low levels. A further example of a receptor modifying T cell functions is CD95, also known as the Fas receptor, which mediates apoptotic signaling by Fas-ligand expressed on the surface of other cells. CD95 has been reported to modulate TCR/CD3-driven signaling pathways in resting T lymphocytes.

An example of a NK cell specific receptor molecule is CD16, a low affinity Fc receptor and NKG2D. An example of a receptor molecule that is present on the surface of both T cells and natural killer (NK) cells is CD2 and further members of the CD2-superfamily. CD2 is able to act as a co-stimulatory molecule on T and NK cells.

In some embodiments, the first binding site of the antibody molecule binds a tumor antigen and the second binding site binds a T cell specific receptor molecule and/or a natural killer (NK) cell specific receptor molecule.

In some embodiments, the first binding site of the antibody molecule binds FSHR, and the second binding site binds a T cell specific receptor molecule and/or a natural killer (NK) cell specific receptor molecule. In some embodiments, the first binding site of the antibody molecule binds FSHR and the second binding site binds one of CD3, TCR, CD28, CD16, NKG2D, Ox40, 4-1BB, CD2, CD5, CD40, FcgRs, FceRs, FcaRs and CD95. In some embodiments, the first binding site of the antibody molecule binds FSHR and the second binding site binds CD3.

In some embodiments, the first binding site of the antibody molecule binds a T cell specific receptor molecule and/or a natural killer (NK) cell specific receptor molecule and the second binding site binds a tumor antigen. In some embodiments, the first binding site of the antibody binds a T cell specific receptor molecule and/or a natural killer (NK) cell specific receptor molecule and the second binding site binds FSHR. In some embodiments, the first binding site of the antibody binds one of CD3, TCR, CD28, CD16, NKG2D, Ox40, 4-1BB, CD2, CD5, CD40, FcgRs, FceRs, FcaRs and CD95, and the second binding site binds FSHR. In some embodiments, the first binding site of the antibody binds CD3, and the second binding site binds FSHR.

In one embodiment the bispecific antibody of the invention comprises a BiTE, comprising one or more scFv antibody fragments as described herein, thereby allowing the BiTE to bind or react with the desired target molecules.

In one embodiment the BiTE, comprises a first scFv specific for binding to a target disease-specific antigen linked to a second scFv specific for binding to a T cell specific receptor molecule. The linkage may place the first and second domains in any order, for example, in one embodiment, a nucleotide sequence encoding a scFv specific for binding to a target disease-specific antigen is oriented C-Terminal to a scFv specific for binding to a T cell specific receptor molecule. In another embodiment, a nucleotide sequence encoding a scFv specific for binding to a target disease-specific antigen is oriented N-Terminal to a nucleotide sequence encoding a scFv specific for binding to a T cell specific receptor molecule.

### Bifunctional Antibody

The bispecific T cell engager can be a bifunctional antibody, a fragment thereof, a variant thereof, or a combination thereof. The bifunctional antibody can bind or react with the antigen described below. The bifunctional antibody can also be modified to impart an additional functionality to the antibody beyond recognition of and binding to the antigen. Such a modification can include, but is not limited to, coupling to factor H or a fragment thereof. Factor H is a soluble regulator of complement activation and thus, may contribute to an immune response via complement-mediated lysis (CML).

### Extension of Antibody Half-Life

The synthetic antibody (e.g., scFv antibody fragment, BICE or BiTE) may be modified to extend or shorten the half-life of the antibody in the subject. The modification may extend or shorten the half-life of the antibody in the serum of the subject.

The modification may be present in a constant region of the antibody. The modification may be one or more amino acid substitutions in a constant region of the antibody that extend the half-life of the antibody as compared to a half-life of an antibody not containing the one or more amino acid substitutions. The modification may be one or more amino acid substitutions in the CH2 domain of the antibody that extend the half-life of the antibody as compared to a half-life of an antibody not containing the one or more amino acid substitutions.

In some embodiments, the one or more amino acid substitutions in the constant region may include replacing a methionine residue in the constant region with a tyrosine residue, a serine residue in the constant region with a threonine residue, a threonine residue in the constant region with a glutamate residue, or any combination thereof, thereby extending the half-life of the antibody.

In other embodiments, the one or more amino acid substitutions in the constant region may include replacing a methionine residue in the CH2 domain with a tyrosine residue, a serine residue in the CH2 domain with a threonine residue, a threonine residue in the CH2 domain with a glutamate residue, or any combination thereof, thereby extending the half-life of the antibody.

### Antigen

In one embodiment, the synthetic antibody (e.g., scFv antibody fragment, BICE or BiTE) or CAR molecule is directed to an antigen or fragment or variant thereof. The antigen can be a nucleic acid sequence, an amino acid sequence, a polysaccharide or a combination thereof. The nucleic acid sequence can be DNA, RNA, cDNA, a variant thereof, a fragment thereof, or a combination thereof. The amino acid sequence can be a protein, a peptide, a variant thereof, a fragment thereof, or a combination thereof. The polysaccharide can be a nucleic acid encoded polysaccharide.

The antigen can be a tumor antigen. The antigen can be associated with increased risk of cancer development or progression. In one embodiment, the antigen can be FSHR.

In one embodiment, a synthetic bispecific immune cell engager of the invention targets two or more antigens. In one embodiment, at least one antigen of a bispecific antibody is a tumor antigen. In one embodiment, at least one antigen of a bispecific antibody is a T-cell activating antigen.

### Tumor Antigen

The antigen binding domain of the synthetic antibody (e.g., scFv antibody fragment, BICE or BiTE) or CAR molecule of the invention can interact with a tumor antigen. In the context of the present invention, “tumor antigen” or “hyperproliferative disorder antigen” or “antigen associated with a hyperproliferative disorder,” refers to antigens that are common to specific hyperproliferative disorders such as cancer. The type of tumor antigen referred to in the invention may be a tumor-specific antigen (TSA) or a tumor-associated antigen (TAA). A TSA is unique to tumor cells and does not occur on other cells in the body. A TAA antigen is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

The antigens discussed herein are merely included by way of example. The list is not intended to be exclusive and further examples will be readily apparent to those of skill in the art.

Tumor antigens are proteins that are produced by tumor cells that elicit an immune response, particularly T-cell mediated immune responses. The selection of the antigen binding moiety of the invention will depend on the particular type of cancer to be treated. Tumor antigens are well known in the art and include, for example, a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

Illustrative examples of a tumor associated surface antigen are CD10, CD19, CD20, CD22, CD33, CD123, B-cell maturation antigen (BCMA), Fms-like tyrosine kinase 3 (FLT-3, CD135), chondroitin sulfate proteoglycan 4 (CSPG4, melanoma-associated chondroitin sulfate proteoglycan), Epidermal growth factor receptor (EGFR), Her2, Her3, IGFR, CD133, IL3R, fibroblast activating protein (FAP), CDCP1, Derlin1, Tenascin, frizzled 1-10, the vascular antigens VEGFR2 (KDR/FLK1), VEGFR3 (FLT4, CD309), PDGFR-α (CD140a), PDGFR-beta. (CD140b) Endoglin, CLEC14, Tem1-8, and Tie2. Further examples may include A33, CAMPATH-1 (CDw52), Carcinoembryonic antigen (CEA), Carboanhydrase IX (MN/CA IX), CD21, CD25, CD30, CD34, CD37, CD44v6, CD45, CD133, de2-7 EGFR, EGFRVIII, EpCAM, Ep-CAM, Folate-binding protein, G250, Fms-like tyrosine kinase 3 (FLT-3, CD135), follicle stimulating hormone receptor (FSHR), c-Kit (CD117), CSFIR (CD115), HLA-DR, IGFR, IL-2 receptor, IL3R, MCSP (Melanoma-associated cell surface chondroitin sulphate proteoglycane), Muc-1, Prostate-specific membrane antigen (PSMA), Prostate stem cell antigen (PSCA), Prostate specific antigen (PSA), and TAG-72. Examples of antigens expressed on the extracellular matrix of tumors are tenascin and the fibroblast activating protein (FAP).

In one embodiment, the tumor antigen is a hormone or fragment thereof which can be used to target a specific receptor. Examples include, but are not limited to, FSH hormone, LH hormone, TSH hormone or fragments thereof.

Non-limiting examples of TSA or TAA antigens include the following: Differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alphafetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

Aspects of the present invention include compositions for enhancing an immune response against an antigen in a subject in need thereof, comprising a synthetic antibody (e.g., scFv antibody fragment, BICE or BiTE) or CAR molecule capable of generating an immune response in the subject, or a biologically functional fragment or variant thereof. In some embodiments, the antigen is FSHR. In some embodiments, the synthetic antibody of this invention is a BiTE comprising an scFv targeting FSHR.

### T Cell Specific Receptor

In one embodiment, the BiTE or BICE of the invention comprises a scFv of a T cell specific receptor. T cell specific receptors include, but are not limited to, CD3, TCR, CD28, CD16, NKG2D, Ox40, 4-1BB, CD2, CD5, CD40, FcgRs, FceRs, FcaRs and CD95.

### CAR Molecules

In one embodiment, the invention provides a chimeric antigen receptor (CAR) comprising an antigen binding domain and a T cell activation domain. In one embodiment, the antigen binding domain is a targeting domain, wherein the targeting domain directs the cell expressing the CAR to a cell or particle expressing the antigen. In one embodiment, the antigen is FSHR.

In various embodiments, the CAR can be a “first generation,” “second generation,” “third generation,” “fourth generation” or “fifth generation” CAR (see, for example, Sadelain et al., Cancer Discov. 3 (4): 388-398 (2013); Jensen et al., Immunol. Rev. 257:127-133 (2014); Sharpe et al., Dis. Model Mech. 8 (4): 337-350 (2015); Brentjens et al., Clin. Cancer Res. 13:5426-5435 (2007); Gade et al., Cancer Res. 65:9080-9088 (2005); Maher et al., Nat. Biotechnol. 20:70-75 (2002); Kershaw et al., J. Immunol. 173:2143-2150 (2004); Sadelain et al., Curr. Opin. Immunol. (2009); Hollyman et al., J. Immunother. 32:169-180 (2009)).

“First generation” CARs for use in the invention comprise an antigen binding domain, for example, a single-chain variable fragment (scFv), fused to a transmembrane domain, which is fused to a cytoplasmic/intracellular domain of the T cell receptor chain. “First generation” CARs typically have the intracellular domain from the CD34-chain, which is the primary transmitter of signals from endogenous T cell receptors (TCRs). “First generation” CARs can provide de novo antigen recognition and cause activation of both CD4+ and CD8+ T cells through their CD3ζ chain signaling domain in a single fusion molecule, independent of HLA-mediated antigen presentation.

“Second-generation” CARs for use in the invention comprise an antigen binding domain, for example, a single-chain variable fragment (scFv), fused to an intracellular signaling domain capable of activating T cells and a co-stimulatory domain designed to augment T cell potency and persistence (Sadelain et al., Cancer Discov. 3:388-398 (2013)). CAR design can therefore combine antigen recognition with signal transduction, two functions that are physiologically borne by two separate complexes, the TCR heterodimer and the CD3 complex. “Second generation” CARs include an intracellular domain from various co-stimulatory molecules, for example, CD28, 4-1BB, ICOS, OX40, and the like, in the cytoplasmic tail of the CAR to provide additional signals to the cell.

“Second generation” CARs provide both co-stimulation, for example, by CD28 or 4-1BB domains, and activation, for example, by a CD3ζ signaling domain. Preclinical studies have indicated that “Second Generation” CARs can improve the anti-tumor activity of T cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL) (Davila et al., Oncoimmunol. 1 (9): 1577-1583 (2012)).

“Third generation” CARs provide multiple co-stimulation, for example, by comprising both CD28 and 4-1BB domains, and activation, for example, by comprising a CD3 activation domain.

“Fourth generation” CARs provide co-stimulation, for example, by CD28 or 4-1BB domains, and activation, for example, by a CD3ζ signaling domain in addition to a constitutive or inducible chemokine component.

“Fifth generation” CARs provide co-stimulation, for example, by CD28 or 4-1BB domains, and activation, for example, by a CD3ζ signaling domain, a constitutive or inducible chemokine component, and an intracellular domain of a cytokine receptor, for example, IL-2Rβ.

In various embodiments, the CAR can be included in a multivalent CAR system, for example, a DualCAR or “TandemCAR” system. Multivalent CAR systems include systems or cells comprising multiple CARs and systems or cells comprising bivalent/bispecific CARs targeting more than one antigen.

In the embodiments disclosed herein, the CARs generally comprise an antigen binding domain, a transmembrane domain and an intracellular domain, as described above. In a particular non-limiting embodiment, the antigen-binding domain is a FSHR scFv antibody fragment, or a variant thereof, specific for binding to FSHR. In some embodiments the FSHR binding domain comprises a sequence as set forth in SEQ ID NO: 3.

In various embodiments, the CAR molecules of the invention provides for both co-stimulation by CD28 and/or 4-1BB domains, and activation, by a CD3ζ signaling domain.

In the embodiments disclosed herein, the CAR comprises a sequence as set forth in SEQ ID NO:4, SEQ ID NO:5 or SEQ ID NO:6 or a fragment or variant thereof.

### Substrates

In one embodiment, the present invention provides a scaffold, substrate, or device comprising a bispecific immune cell engager, fragment thereof, or nucleic acid molecule encoding the same. For example, in some embodiments, the present invention provides a tissue engineering scaffold, including but not limited to, a hydrogel, electrospun scaffold, polymeric matrix, or the like, comprising the modulator. In certain embodiments, a bispecific immune cell engager, fragment thereof, or nucleic acid molecule encoding the same, may be coated along the surface of the scaffold, substrate, or device. In certain embodiments, the bispecific immune cell engager, fragment thereof, or nucleic acid molecule encoding the same is encapsulated within the scaffold, substrate, or device.

### Recombinant Nucleic Acid Sequence

As described above, the composition can comprise a recombinant nucleic acid sequence. The recombinant nucleic acid sequence can encode the synthetic antibody (e.g., scFv antibody fragment, BICE or BiTE) or the CAR molecule, a fragment thereof, a variant thereof, or a combination thereof.

The recombinant nucleic acid sequence can be a heterologous nucleic acid sequence. The recombinant nucleic acid sequence can include at least one heterologous nucleic acid sequence or one or more heterologous nucleic acid sequences.

In one embodiment, a nucleotide sequence encoding a FSHR-BiTE comprises a nucleotide sequence of SEQ ID NO: 1 or a fragment or variant thereof.

The recombinant nucleic acid sequence can be an optimized nucleic acid sequence. Such optimization can increase or alter the immunogenicity of the antibody. Optimization can also improve transcription and/or translation. Optimization can include one or more of the following: low GC content leader sequence to increase transcription: mRNA stability and codon optimization; addition of a kozak sequence (e.g., GCC ACC) for increased translation; addition of an immunoglobulin (Ig) leader sequence encoding a signal peptide; and eliminating to the extent possible cis-acting sequence motifs (i.e., internal TATA boxes).

The recombinant nucleic acid sequence can include one or more recombinant nucleic acid sequence constructs. The recombinant nucleic acid sequence construct can include one or more components, which are described in more detail below.

The recombinant nucleic acid sequence construct can include a heterologous nucleic acid sequence that encodes a heavy chain polypeptide, a fragment thereof, a variant thereof, or a combination thereof. The recombinant nucleic acid sequence construct can include a heterologous nucleic acid sequence that encodes a light chain polypeptide, a fragment thereof, a variant thereof, or a combination thereof. The recombinant nucleic acid sequence construct can also include a heterologous nucleic acid sequence that encodes a protease or peptidase cleavage site. The recombinant nucleic acid sequence construct can also include a heterologous nucleic acid sequence that encodes an internal ribosome entry site (IRES). An IRES may be either a viral IRES or an eukaryotic IRES. The recombinant nucleic acid sequence construct can include one or more leader sequences, in which each leader sequence encodes a signal peptide. The recombinant nucleic acid sequence construct can include one or more promoters, one or more introns, one or more transcription termination regions, one or more initiation codons, one or more termination or stop codons, and/or one or more polyadenylation signals. The recombinant nucleic acid sequence construct can also include one or more linker or tag sequences. The tag sequence can encode a hemagglutinin (HA) tag.

### Promoter

The recombinant nucleic acid sequence construct can include one or more promoters. The one or more promoters may be any promoter that is capable of driving gene expression and regulating gene expression. Such a promoter is a cis-acting sequence element required for transcription via a DNA dependent RNA polymerase. Selection of the promoter used to direct gene expression depends on the particular application. The promoter may be positioned about the same distance from the transcription start in the recombinant nucleic acid sequence construct as it is from the transcription start site in its natural setting. However, variation in this distance may be accommodated without loss of promoter function.

The promoter may be operably linked to the heterologous nucleic acid sequence encoding the heavy chain polypeptide and/or light chain polypeptide. The promoter may be a promoter shown effective for expression in eukaryotic cells. The promoter operably linked to the coding sequence may be a CMV promoter, a promoter from simian virus 40 (SV40), such as SV40 early promoter and SV40 later promoter, a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human actin, human myosin, human hemoglobin, human muscle creatine, human polyhedrin, or human metalothionein.

The promoter can be a constitutive promoter or an inducible promoter, which initiates transcription only when the host cell is exposed to some particular external stimulus. In the case of a multicellular organism, the promoter can also be specific to a particular tissue or organ or stage of development. The promoter may also be a tissue specific promoter, such as a muscle or skin specific promoter, natural or synthetic. Examples of such promoters are described in US patent application publication no. US20040175727, the contents of which are incorporated herein in its entirety.

The promoter can be associated with an enhancer. The enhancer can be located upstream of the coding sequence. The enhancer may be human actin, human myosin, human hemoglobin, human muscle creatine or a viral enhancer such as one from CMV, FMDV, RSV or EBV. Polynucleotide function enhances are described in U.S. Pat. Nos. 5,593,972, 5,962,428, and WO94/016737, the contents of each are fully incorporated by reference.

### Transcription Termination Region

The recombinant nucleic acid sequence construct can include one or more transcription termination regions. The transcription termination region can be downstream of the coding sequence to provide for efficient termination. The transcription termination region can be obtained from the same gene as the promoter described above or can be obtained from one or more different genes.

### Initiation Codon

The recombinant nucleic acid sequence construct can include one or more initiation codons. The initiation codon can be located upstream of the coding sequence. The initiation codon can be in frame with the coding sequence. The initiation codon can be associated with one or more signals required for efficient translation initiation, for example, but not limited to, a ribosome binding site.

### Termination Codon

The recombinant nucleic acid sequence construct can include one or more termination or stop codons. The termination codon can be downstream of the coding sequence. The termination codon can be in frame with the coding sequence. The termination codon can be associated with one or more signals required for efficient translation termination.

### Polyadenylation Signal

The recombinant nucleic acid sequence construct can include one or more polyadenylation signals. The polyadenylation signal can include one or more signals required for efficient polyadenylation of the transcript. The polyadenylation signal can be positioned downstream of the coding sequence. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 plasmid (Invitrogen, San Diego, CA).

### Protease Cleavage Site

The recombinant nucleic acid sequence construct can include the heterologous nucleic acid sequence encoding the protease cleavage site. The protease cleavage site can be recognized by a protease or peptidase. The protease can be an endopeptidase or endoprotease, for example, but not limited to, furin, elastase, HtrA, calpain, trypsin, chymotrypsin, trypsin, and pepsin. The protease can be furin. In other embodiments, the protease can be a serine protease, a threonine protease, cysteine protease, aspartate protease, metalloprotease, glutamic acid protease, or any protease that cleaves an internal peptide bond (i.e., does not cleave the N-terminal or C-terminal peptide bond).

The protease cleavage site can include one or more amino acid sequences that promote or increase the efficiency of cleavage. The one or more amino acid sequences can promote or increase the efficiency of forming or generating discrete polypeptides. The one or more amino acids sequences can include a 2A peptide sequence.

### Vector

The recombinant nucleic acid sequence construct described above can be placed in one or more vectors. The one or more vectors can contain an origin of replication. The one or more vectors can be a plasmid, bacteriophage, bacterial artificial chromosome or yeast artificial chromosome. The one or more vectors can be either a self-replication extra chromosomal vector, or a vector which integrates into a host genome.

The one or more vectors can be a heterologous expression construct, which is generally a plasmid that is used to introduce a specific gene into a target cell. Once the expression vector is inside the cell, the heavy chain polypeptide and/or light chain polypeptide that are encoded by the recombinant nucleic acid sequence construct is produced by the cellular-transcription and translation machinery ribosomal complexes. The one or more vectors can express large amounts of stable messenger RNA, and therefore proteins.

### Expression Vector

The one or more vectors can be a circular plasmid or a linear nucleic acid. The circular plasmid and linear nucleic acid are capable of directing expression of a particular nucleotide sequence in an appropriate subject cell. The one or more vectors comprising the recombinant nucleic acid sequence construct may be chimeric, meaning that at least one of its components is heterologous with respect to at least one of its other components.

### Plasmid

The one or more vectors can be a plasmid. The plasmid may be useful for transfecting cells with the recombinant nucleic acid sequence construct. The plasmid may be useful for introducing the recombinant nucleic acid sequence construct into the subject. The plasmid may also comprise a regulatory sequence, which may be well suited for gene expression in a cell into which the plasmid is administered.

The plasmid may also comprise a mammalian origin of replication in order to maintain the plasmid extrachromosomally and produce multiple copies of the plasmid in a cell. The plasmid may be pVAX1, pCEP4 or pREP4 from Invitrogen (San Diego, CA), which may comprise the Epstein Barr virus origin of replication and nuclear antigen EBNA-1 coding region, which may produce high copy episomal replication without integration. The backbone of the plasmid may be pAV0242. The plasmid may be a replication defective adenovirus type 5 (Ad5) plasmid.

The plasmid may be pSE420 (Invitrogen, San Diego, Calif.), which may be used for protein production in Escherichia coli (E. coli). The plasmid may also be p YES2 (Invitrogen, San Diego, Calif.), which may be used for protein production in Saccharomyces cerevisiae strains of yeast. The plasmid may also be of the MAXBAC™ complete baculovirus expression system (Invitrogen, San Diego, Calif.), which may be used for protein production in insect cells. The plasmid may also be pcDNAI or pcDNA3 (Invitrogen, San Diego, Calif.), which may be used for protein production in mammalian cells such as Chinese hamster ovary (CHO) cells.

RNA

In one embodiment, the nucleic acid is an RNA molecule. In one embodiment, the RNA molecule is transcribed from a DNA sequence. Accordingly, in one embodiment, the invention provides an RNA molecule encoding one or more of the synthetic antibodies of the invention. The RNA may be plus-stranded. Accordingly, in some embodiments, the RNA molecule can be translated by cells without needing any intervening replication steps such as reverse transcription. A RNA molecule useful with the invention may have a 5′ cap (e.g. a 7-methylguanosine). This cap can enhance in vivo translation of the RNA. The 5′ nucleotide of a RNA molecule useful with the invention may have a 5′ triphosphate group. In a capped RNA this may be linked to a 7-methylguanosine via a 5′-to-5′ bridge. A RNA molecule may have a 3′ poly-A tail. It may also include a poly-A polymerase recognition sequence (e.g. AAUAAA) near its 3′ end. A RNA molecule useful with the invention may be single-stranded. A RNA molecule useful with the invention may comprise synthetic RNA. In some embodiments, the RNA molecule is a naked RNA molecule. In one embodiment, the RNA molecule is comprised within a vector.

In one embodiment, the RNA has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of RNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many RNAs is known in the art. In other embodiments, the 5′ UTR can be derived from an RNA virus whose RNA genome is stable in cells. In other embodiments, various nucleotide analogues can be used in the 3′ or 5′ UTR to impede exonuclease degradation of the RNA.

In one embodiment, the RNA has both a cap on the 5′ end and a 3′ poly(A) tail which determine ribosome binding, initiation of translation and stability of RNA in the cell.

In one embodiment, the RNA is a nucleoside-modified RNA. Nucleoside-modified RNA have particular advantages over non-modified RNA, including for example, increased stability, low or absent innate immunogenicity, and enhanced translation.

### Circular and Linear Vector

The one or more vectors may be circular plasmid, which may transform a target cell by integration into the cellular genome or exist extrachromosomally (e.g., autonomous replicating plasmid with an origin of replication). The vector can be pVAX, pcDNA3.0, or provax, or any other expression vector capable of expressing the heavy chain polypeptide and/or light chain polypeptide encoded by the recombinant nucleic acid sequence construct.

Also provided herein is a linear nucleic acid, or linear expression cassette (“LEC”), that is capable of being efficiently delivered to a subject and expressing the heavy chain polypeptide and/or light chain polypeptide encoded by the recombinant nucleic acid sequence construct. The LEC may be any linear DNA devoid of any phosphate backbone. The LEC may not contain any antibiotic resistance genes and/or a phosphate backbone. The LEC may not contain other nucleic acid sequences unrelated to the desired gene expression.

The LEC may be derived from any plasmid capable of being linearized. The plasmid may be capable of expressing the heavy chain polypeptide and/or light chain polypeptide encoded by the recombinant nucleic acid sequence construct. The plasmid can be pNP (Puerto Rico/34) or pM2 (New Caledonia/99). The plasmid may be WLV009, pVAX, pcDNA3.0, or provax, or any other expression vector capable of expressing the heavy chain polypeptide and/or light chain polypeptide encoded by the recombinant nucleic acid sequence construct.

The LEC can be pcrM2. The LEC can be pcrNP. pcrNP and pcrMR can be derived from pNP (Puerto Rico/34) and pM2 (New Caledonia/99), respectively.

### Method of Preparing the Vector

Provided herein is a method for preparing the one or more vectors in which the recombinant nucleic acid sequence construct has been placed. After the final subcloning step, the vector can be used to inoculate a cell culture in a large scale fermentation tank, using known methods in the art.

The one or more vectors can be formulated or manufactured using a combination of known devices and techniques, but preferably they are manufactured using a plasmid manufacturing technique that is described in a licensed, co-pending U.S. provisional application U.S. Ser. No. 60/939,792, which was filed on May 23, 2007. In some examples, the DNA plasmids described herein can be formulated at concentrations greater than or equal to 10 mg/mL. The manufacturing techniques also include or incorporate various devices and protocols that are commonly known to those of ordinary skill in the art, in addition to those described in U.S. Ser. No. 60/939,792, including those described in a licensed patent, U.S. Pat. No. 7,238,522, which issued on Jul. 3, 2007. The above-referenced application and patent, U.S. Ser. No. 60/939,792 and U.S. Pat. No. 7,238,522, respectively, are hereby incorporated in their entirety.

Expression from the Recombinant Nucleic Acid Sequence Construct

As described above, the recombinant nucleic acid sequence construct can include, amongst the one or more components, the heterologous nucleic acid sequence encoding the heavy chain polypeptide and/or the heterologous nucleic acid sequence encoding the light chain polypeptide. Accordingly, the recombinant nucleic acid sequence construct can facilitate expression of the heavy chain polypeptide and/or the light chain polypeptide.

When arrangement 1 as described above is utilized, the first recombinant nucleic acid sequence construct can facilitate the expression of the heavy chain polypeptide and the second recombinant nucleic acid sequence construct can facilitate expression of the light chain polypeptide. When arrangement 2 as described above is utilized, the recombinant nucleic acid sequence construct can facilitate the expression of the heavy chain polypeptide and the light chain polypeptide.

Upon expression, for example, but not limited to, in a cell, organism, or mammal, the heavy chain polypeptide and the light chain polypeptide can assemble into the synthetic antibody (e.g., scFv antibody fragment, BICE or BiTE) or the CAR molecule. In particular, the heavy chain polypeptide and the light chain polypeptide can interact with one another such that assembly results in the synthetic antibody (e.g., scFv antibody fragment, BICE or BiTE) or the CAR molecule being capable of binding the antigen. In other embodiments, the heavy chain polypeptide and the light chain polypeptide can interact with one another such that assembly results synthetic antibody (e.g., scFv antibody fragment, BICE or BiTE) or the CAR molecule being more immunogenic as compared to an antibody not assembled as described herein. In still other embodiments, the heavy chain polypeptide and the light chain polypeptide can interact with one another such that assembly results in the synthetic antibody (e.g., scFv antibody fragment, BICE or BiTE) or the CAR molecule being capable of eliciting or inducing an immune response against the antigen.

### Excipients and Other Components of the Composition

The composition may further comprise a pharmaceutically acceptable excipient. The pharmaceutically acceptable excipient can be functional molecules such as vehicles, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and the poly-L-glutamate may be present in the composition at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the composition. The composition may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. The transfection facilitating agent is a polyanion, polycation, including poly-L-glutamate (LGS), or lipid. Concentration of the transfection agent in the composition is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The composition can be formulated according to the mode of administration to be used. An injectable pharmaceutical composition can be sterile, pyrogen free and particulate free. An isotonic formulation or solution can be used. Additives for isotonicity can include sodium chloride, dextrose, mannitol, sorbitol, and lactose. The composition can comprise a vasoconstriction agent. The isotonic solutions can include phosphate buffered saline. The composition can further comprise stabilizers including gelatin and albumin. The stabilizers can allow the formulation to be stable at room or ambient temperature for extended periods of time, including LGS or polycations or polyanions.

### Methods of Delivery of the Composition

The present invention also relates to a method of delivering the composition to the subject in need thereof. The method of delivery can include, administering the composition to the subject. In some embodiments, the present invention relates to administration of a bispecific antibody of the invention, scFv, CAR molecule, CAR T cell, or a nucleic acid molecule encoding a bispecific antibody, scFv, or CAR molecule of the invention. In some embodiments, the nucleic acid molecule is a DNA molecule. In some embodiments, the nucleic acid molecule is an RNA molecule. In some embodiments, the nucleic acid molecule is an mRNA molecule.

Administration can include, but is not limited to, intravenous delivery of an antibody, scFv, CAR molecule, or CAR T cell, DNA injection, liposome mediated delivery, and nanoparticle facilitated delivery.

The mammal receiving delivery of the composition may be human, primate, non-human primate, cow, cattle, sheep, goat, antelope, bison, water buffalo, bison, bovids, deer, hedgehogs, elephants, llama, alpaca, mice, rats, and chicken.

The composition may be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian can readily determine the dosing regimen and route of administration that is most appropriate for a particular animal. The composition may be administered by traditional syringes, needleless injection devices, “microprojectile bombardment gone guns”, or other physical methods such as electroporation (“EP”), “hydrodynamic method”, or ultrasound.

### Method of Treatment

Also provided herein is a method of treating, protecting against, and/or preventing disease in a subject in need thereof by administration of a bispecific antibody of the invention, scFv, CAR molecule, CAR T cell, or a nucleic acid molecule encoding a bispecific antibody, scFv, or CAR molecule of the invention to the subject. The method can include administering a composition comprising a bispecific antibody of the invention, scFv, CAR molecule, CAR T cell, or a nucleic acid molecule encoding a bispecific antibody, scFv, or CAR molecule of the invention to the subject. Administration of the composition to the subject can be done using the method of delivery described above.

In certain embodiments, the invention provides a method of treating protecting against, and/or preventing cancer. In one embodiment, the method treats, protects against, and/or prevents tumor growth. In one embodiment, the method treats, protects against, and/or prevents cancer progression. In one embodiment, the method treats, protects against, and/or prevents cancer metastasis.

In one embodiment, the invention provides methods for preventing growth of benign tumors, such as, but not limited to, uterine fibroids. The methods comprise administering an effective amount of one or more of the compositions of the invention to a subject diagnosed with a benign tumor.

Upon administration of the synthetic antibody, BiTe, scFv, CAR molecule or CAR T cell to the subject, the synthetic antibody, BiTe, scFv, CAR molecule or CAR T cell can bind to or react with the antigen. Such binding can neutralize the antigen, block recognition of the antigen by another molecule, for example, a protein or nucleic acid, and elicit or induce an immune response to the antigen, thereby treating, protecting against, and/or preventing the disease associated with the antigen in the subject.

The composition dose can be between 1 μg to 10 mg active component/kg body weight/time, and can be 20 μg to 10 mg component/kg body weight/time. The composition can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of composition doses for effective treatment can be 1, 2, 3, 4, 5, 6, 7, 8, 9, or 10.

### Cancer Therapy

The invention provides methods of treating or preventing cancer, or of treating and preventing growth or metastasis of tumors. Related aspects of the invention provide methods of preventing, aiding in the prevention, and/or reducing metastasis of hyperplastic or tumor cells in an individual.

One aspect of the invention provides a method of inhibiting metastasis in an individual in need thereof, the method comprising administering to the individual an effective amount of a composition of the invention. The invention further provides a method of inhibiting metastasis in an individual in need thereof, the method comprising administering to the individual an effective metastasis-inhibiting amount of any one of the compositions described herein.

In some embodiments of treating or preventing cancer, or of treating and preventing metastasis of tumors in an individual in need thereof, a second agent is administered to the individual, such as an antineoplastic agent. In some embodiments, the second agent comprises a second metastasis-inhibiting agent, such as a plasminogen antagonist, or an adenosine deaminase antagonist. In other embodiments, the second agent is an angiogenesis inhibiting agent.

The compositions of the invention can be used to prevent, abate, minimize, control, and/or lessen cancer in humans and animals. The compositions of the invention can also be used to slow the rate of primary tumor growth. The compositions of the invention when administered to a subject in need of treatment can be used to stop the spread of cancer cells. As such, the compositions of the invention can be administered as part of a combination therapy with one or more drugs or other pharmaceutical agents. When used as part of the combination therapy, the decrease in metastasis and reduction in primary tumor growth afforded by the compositions of the invention allows for a more effective and efficient use of any pharmaceutical or drug therapy being used to treat the patient. In addition, control of metastasis by the compositions of the invention affords the subject a greater ability to concentrate the disease in one location.

In one embodiment, the invention provides methods for preventing metastasis of malignant tumors or other cancerous cells as well as to reduce the rate of tumor growth. The methods comprise administering an effective amount of one or more of the compositions of the invention to a subject diagnosed with a malignant tumor or cancerous cells or to a subject having a tumor or cancerous cells.

The following are non-limiting examples of cancers that can be treated by the methods and compositions of the invention: ovarian cancer, breast cancer, prostate cancer, renal cancer, colo-rectal cancer, stomach cancer, lung cancer, testicular cancer, endometrial cancer, and thyroid cancer.

In one embodiment, the invention provides a method to treat cancer metastasis comprising treating the subject prior to, concurrently with, or subsequently to the treatment with a composition of the invention, with a complementary therapy for the cancer, such as surgery, chemotherapy, chemotherapeutic agent, radiation therapy, or hormonal therapy or a combination thereof.

Chemotherapeutic agents include cytotoxic agents (e.g., 5-fluorouracil, cisplatin, carboplatin, methotrexate, daunorubicin, doxorubicin, vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxy doxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

Antiproliferative agents are compounds that decrease the proliferation of cells. Antiproliferative agents include alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene and roloxifene), Additional examples of specific antiproliferative agents include, but are not limited to levamisole, gallium nitrate, granisetron, sargramostim strontium-89 chloride, filgrastim, pilocarpine, dexrazoxane, and ondansetron.

The compounds of the invention can be administered alone or in combination with other anti-tumor agents, including cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the invention include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the compositions of the invention include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to; 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In one embodiment, the anti-cancer drug is 5-fluorouracil, taxol, or leucovorin.

Agents that Improve T Cell Response

In some aspects, the synthetic antibody, scFv, BiTE, CAR construct or CAR T cell is administered in combination with at least one agent that improves T cell response. In some embodiments, at least one agent that improves T cell response is an immune checkpoint inhibitor. In some aspects, the immune check point inhibitor used in any one of the methods disclosed herein is an antibody that binds to, and/or reduces or blocks the function of a protein involved in an immune checkpoint pathway.

In some aspects, the immune checkpoint inhibitor targets cytotoxic T-lymphocyte antigen-4 (CTLA-4). In some aspects, the immune checkpoint inhibitor that targets CTLA-4 is Ipilimumab or tremelimumab (ticilimumab, CP-675,206). In some aspects, the immune checkpoint inhibitor targets PD-1. In some aspects, the immune checkpoint inhibitor that targets PD-1 is nivolumab (ONO-4538/BMS-936558, MDX1106, OPDIVOR), pembrolizumab (MK-3475, KEYTRUDAR), pidilizumab (CT-011), atezolizumab (MPDL3280A), cemiplimab (LIBTAYO™), Spartalizumab (PDR001), Camrelizumab (SHR1210), Sintilimab (IBI308), Tislelizumab (BGB-A317), Toripalimab (JS 001), AMP-224, AMP-514 or Spartalizumab (PDR001). In some aspects, the immune checkpoint inhibitor targets PD-L-1. In some aspects, the immune checkpoint inhibitor that targets PD-L-1 is Avelumab, Atezolizumab, Durvalumab, KN035, CK-301, AUNP12, CA-170, or BMS-986189. In some aspects, the immune checkpoint inhibitor targets T cell immunoglobulin and mucin-domain containing-3 (TIM-3) and/or Lymphocyte Activating 3 (LAG3) proteins. In some aspects, the immune checkpoint inhibitor targeting TIM3 is MBG453; TSR-022; or LY3321367. In some aspects, the immune checkpoint inhibitor targeting LAG3 is IMP321 (Eftilagimod alpha), BMS-986016 (Relatlimab), LAG525 (anti-LAG-3 mAb), REGN3767 (anti-LAG-3 mAb), TSR-033 (anti-LAG-3 mAb), MGD013 (a PD-1/LAG-3 bispecific DARTR protein), or FS118 (a LAG-3/PD-L1 bispecific antibody).

In some aspects, the immune checkpoint inhibitor targets indoleamine 2,3-dioxygenase-1 (IDO1). some aspects, the immune checkpoint inhibitor that targets IDO1 is Indoximod (D-1MT; NLG-8189), Navoximod (NLG-919), Epacadostat (INCB024360), BMS-986205, PF-06840003, IOM2983, or RG-70099.

In some aspects, the immune checkpoint inhibitor targets V-domain Ig suppressor of T cell activation (VISTA).

In some aspects, the at least one agent that improves T cell response is a cytokine, such as an inflammatory cytokine. In some aspects, the cytokine is type I IFN or IL-12. In some aspects, the cytokine is a cytokine that shares the common gamma chain receptor. Non-limiting examples of a cytokine that shares the common gamma chain receptor include IL-2, IL-7, IL-15, and IL-21. In some aspects, the at least one agent that improves T cell response is IL-2.

In some aspects, the at least one agent that improves T cell response targets Treg cells. In some aspects, the agent that target Treg cells is an anti-CCR4 antibody, an neuropilin-1 (Nrp-1) inhibitor, or a semaphoring-4a (Sema4a) inhibitor. In some aspects, the at least one agent that improves T cell response is an mTOR inhibitor. Non-limiting examples of an mTOR inhibitor include rapamycin, temsirolimus (CCI-779), everolimus (RAD001), ridaforolimus (AP-23573), sirolimus, dactolisib, BGT226, SF1126, PKI-587, sapanisertib, AZD8055, and AZD2014.

The mode of administration of the immune checkpoint inhibitor is not limited, and may be any mode that is recommended for, or known to be suitable for, the immune checkpoint inhibitor, as described in the art. The mode of administration may vary depending on the particular immune checkpoint inhibitor that is used. In some aspects, the immune checkpoint inhibitor disclosed herein may be administered via a systemic route, such as for example, parenteral routes of administration; a mucosal route; a transdermal route; or directly into a specific tissue. In some aspects, the immune checkpoint inhibitor is administered to a subject in need thereof at a therapeutically effective dose. The therapeutically effective dose depends on factors such as the type of cancer being treated, the age, weight and health of the subject, and route of administration.

The amount of immune checkpoint inhibitor administered to the subject is not limited, and may be any amount as determined by the physician and/or as described or known in the art. In some aspects, the amount of immune checkpoint inhibitor administered to the subject is in the range of about 1 μg/kg to about 50 mg/kg, such as, for example, 5 μg/kg, 10 μg/kg, 50 μg/kg, 100 μg/kg, 500 μg/kg, 1 mg/kg, 2 mg/kg, 3 mg/kg, 4 mg/kg, 5 mg/kg, 10 mg/kg, 15 mg/kg, 20 mg/kg. 25 mg/kg, 30 mg/kg, 35 mg/kg, 40 mg/kg, 45 mg/kg, or 50 mg/kg, including all values and subranges that lie therebetween. In some aspects, the dose may be administered in a volume of about 0.1 mL to about 1.5 mL, for example, about 0.2 mL, about 0.4 mL, 0.5 mL, about 0.6 mL, about 0.8 mL, about 1 mL, or about 1.2 mL, including all values and subranges that lie therebetween.

In some aspects, the immune checkpoint inhibitor is administered concurrently with the synthetic antibody, scFv, BiTE, CAR construct or CAR T cell disclosed herein. In some aspects, the immune checkpoint inhibitor is administered prior to the synthetic antibody, scFv, BiTE, CAR construct or CAR T cell disclosed herein. In some aspects, the immune checkpoint inhibitor is administered after a synthetic antibody, scFv, BITE, CAR construct or CAR T cell disclosed herein.

### Generation of Synthetic Antibodies In Vitro and Ex Vivo

In one embodiment, the synthetic antibody, scFv, BiTE, CAR construct or CAR T cell is generated in vitro or ex vivo. For example, in one embodiment, a nucleic acid encoding a synthetic antibody, scFv, CAR construct or BiTE can be introduced and expressed in an in vitro or ex vivo cell. Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

### Delivery Vehicles

In one embodiment, the present invention provides a composition comprising a delivery vehicle comprising a bispecific anti-FSHR immune cell engaging antibody, scFv, CAR molecule or fragment thereof, or nucleic acid molecule encoding the same, as described herein. In one embodiment, the nucleic acid molecule encoding the bispecific anti-FSHR immune cell engaging antibody scFv, CAR molecule or fragment thereof comprises an mRNA molecule.

Exemplary delivery vehicles include, but are not limited to, microspheres, microparticles, nanoparticles, polymerosomes, liposomes, and micelles. For example, in some embodiments, the delivery vehicle is a lipid nanoparticle loaded with a nucleic acid molecule encoding a bispecific anti-FSHR immune cell engaging antibody of the invention or a fragment thereof. In one embodiment, the nucleic acid molecule encoding the bispecific anti-FSHR immune cell engaging antibody comprises an mRNA molecule. In one embodiment, the mRNA encoding the bispecific anti-FSHR immune cell engaging antibody corresponds to, or is transcribed from, the DNA sequence set forth in SEQ ID NO: 1. In one embodiment, the mRNA encoding the bispecific anti-FSHR immune cell engaging antibody encodes SEQ ID NO:2. In one embodiment, the mRNA encoding the anti-FSHR scFv encodes SEQ ID NO:3.

In some embodiments, the delivery vehicle provides for controlled release, delayed release, or continual release of its loaded cargo. In some embodiments, the delivery vehicle comprises a targeting moiety that targets the delivery vehicle to a treatment site.

In certain instances, expressing a protein by delivering the encoding mRNA has many benefits over methods that use protein, plasmid DNA or viral vectors. During mRNA transfection, the coding sequence of the desired protein is the only substance delivered to cells, thus avoiding all the side effects associated with plasmid backbones, viral genes, and viral proteins. More importantly, unlike DNA- and viral-based vectors, the mRNA does not carry the risk of being incorporated into the genome and protein production starts immediately after mRNA delivery. For example, high levels of circulating proteins have been measured within 15 to 30 min of in vivo injection of the encoding mRNA. In certain embodiments, using mRNA rather than the protein also has many advantages. Half-lives of proteins in the circulation are often short, thus protein treatment would need frequent dosing, while mRNA provides a template for continuous protein production for several days. Purification of proteins is problematic and they can contain aggregates and other impurities that cause adverse effects (Kromminga and Schellekens, 2005, Ann NY Acad Sci 1050:257-265).

In order to confirm the presence of the mRNA sequence in the host cell, a variety of assays may be performed. Such assays include, for example, “molecular biological” assays well known to those of skill in the art, such as Northern blotting and RT-PCR; “biochemical” assays, such as detecting the presence or absence of a particular peptide, e.g., by immunogenic means (ELISAs and Western blots) or by assays described herein to identify agents falling within the scope of the invention.

### Methods of Delivery Using Engineered Immune Cells

In various embodiments, the invention relates to a composition comprising an immune cell engineered for expression of a CAR molecule targeting a tumor cell. Examples of immune cells that can be engineered for expression of a CAR molecule targeting a tumor cell of the invention include, but are not limited to, T cells, B cells, natural killer (NK) cells, or macrophages. In some embodiments, the immune cell further comprises a chimeric antigen receptor (CAR). Therefore, in some embodiments, the invention relates to the use of CAR T-cells for expression or delivery of a FSHR-specific CAR molecule of the invention.

In one embodiment, the present invention provides a method for delivery of a bispecific anti-FSHR immune cell engaging antibody to a target cell providing an engineered immune cell expressing the bispecific anti-FSHR immune cell engaging antibody. In one embodiment, the immune cell is engineered for endogenous secretion of the bispecific anti-FSHR immune cell engaging antibody. In one embodiment, the immune cell is engineered for surface expression of the bispecific anti-FSHR immune cell engaging antibody.

In various embodiments, the invention relates to compositions for endogenous secretion of a T cell-redirecting bispecific antibody (T-bsAb) by engineered T cells (STAb-T cells), which have been engineered to express the bispecific anti-FSHR immune cell engaging antibody. In various embodiments, the method comprises administering to a subject in need thereof a composition comprising a STAb-T cell, wherein the STAb-T cell has been engineered to express the bispecific anti-FSHR immune cell engaging antibody. In some embodiments, the STAb-T cell further comprises a chimeric antigen receptor (CAR). Therefore, in some embodiments, the invention relates to the use of CAR T-cells for expression or delivery of a bispecific anti-FSHR immune cell engaging antibody.

### EXAMPLES

The present invention is further illustrated in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1: Preliminary Studies of DNA Encoded Bispecific T-Cell Engager Targeting FSHR

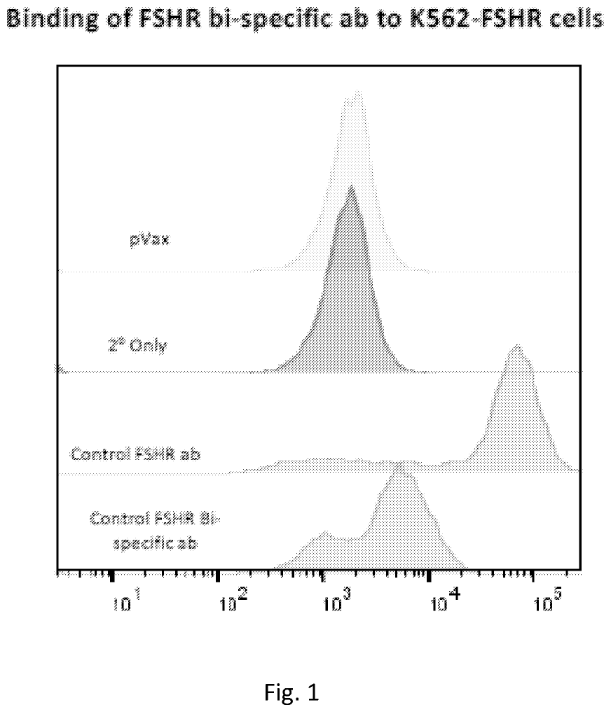

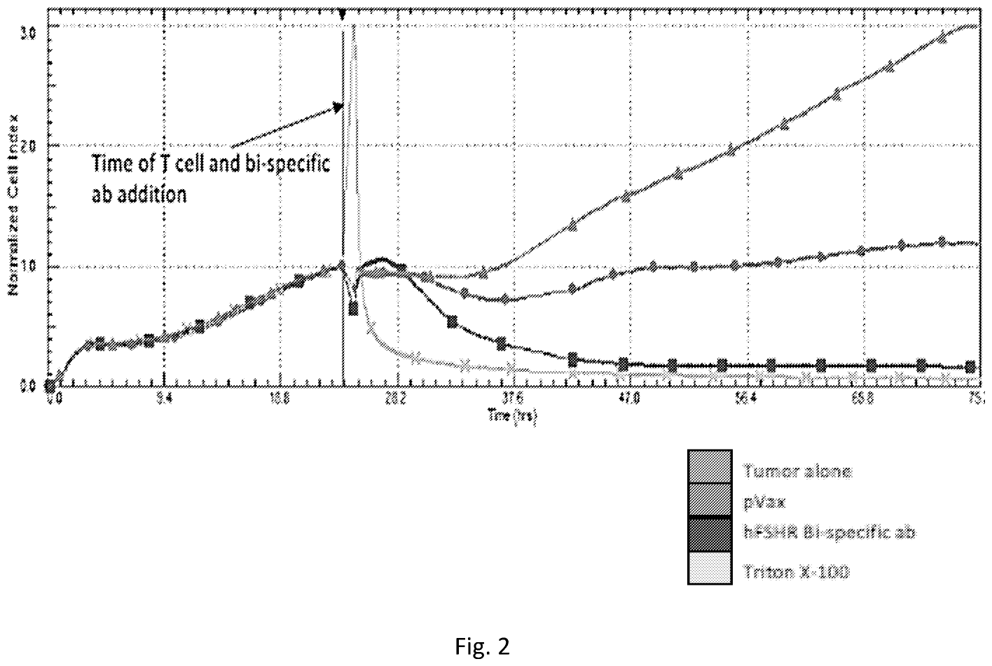

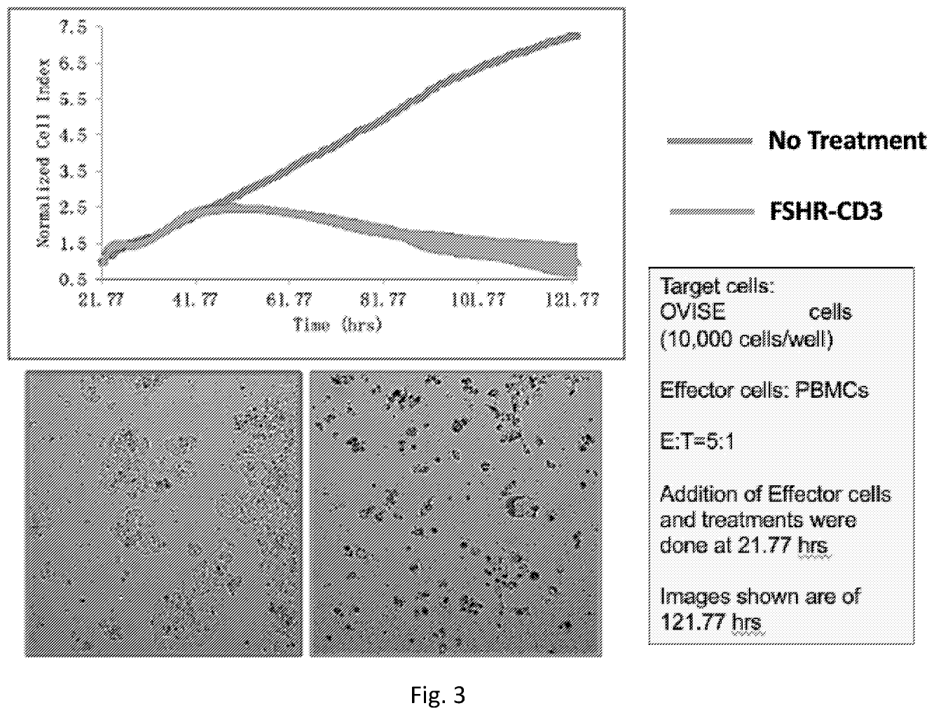

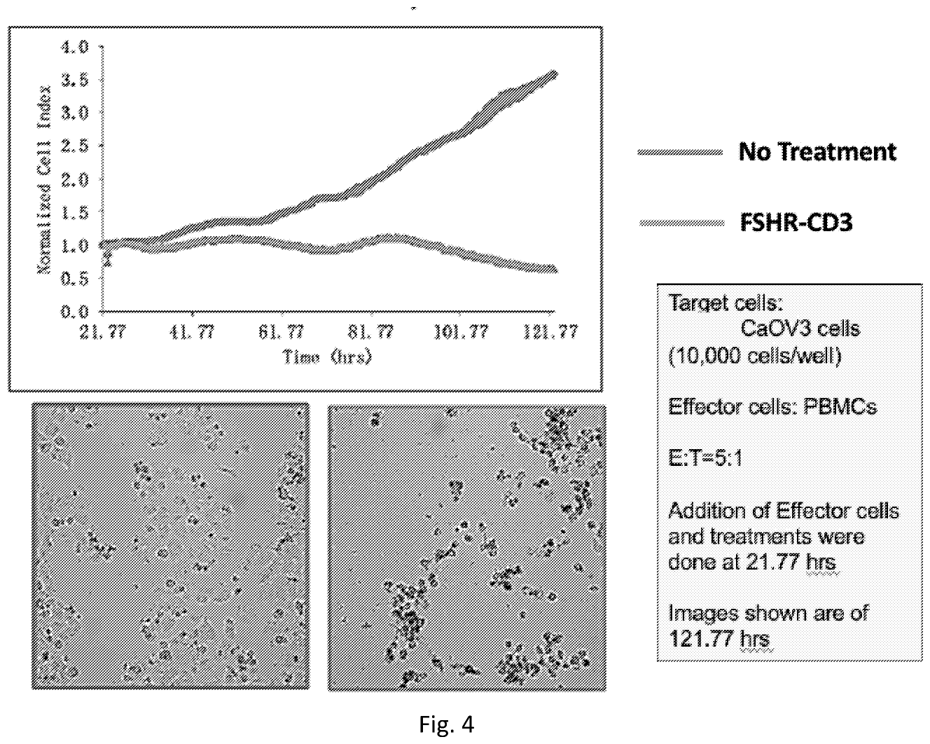

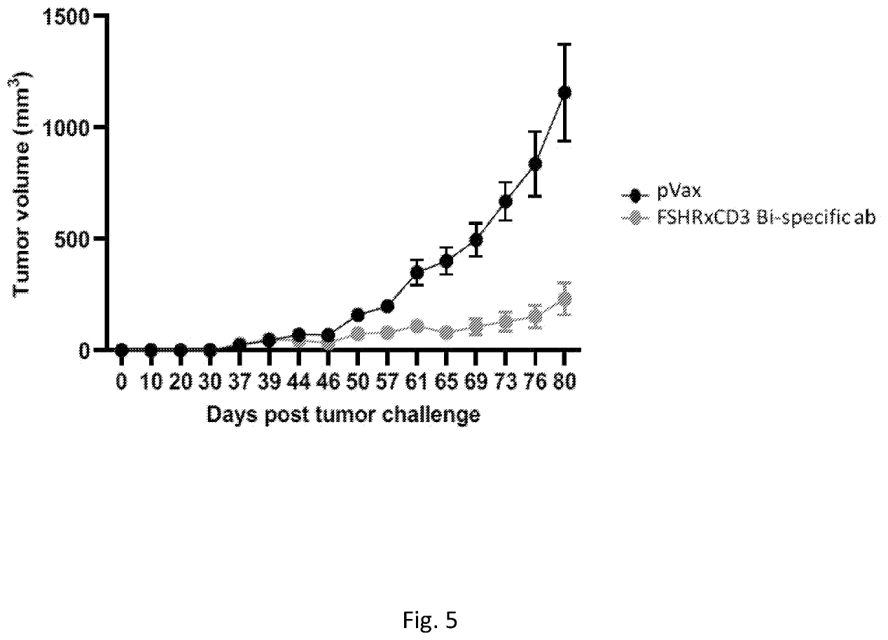

The studies presented herein demonstrate the development of bispecific T-cell engagers targeting FSHR. The FSHR bi-specific antibody binds to K562 cells transfected with human FSHR (FIG. 1) and promotes killing of Ovcar3 cells in the presence of T cells in vitro (FIG. 2). FIG. 3 and FIG. 4 demonstrate the killing response of FSHRxCD3 bi-specific evaluated by xCelligence. In vivo launched FSHRxCD3 bi-specific controls the growth of Ovcar3 tumor cells in NSG mice (FIG. 5). These data demonstrate the development of a new FSHR T cell engaging bispecific which exhibits potent in vitro and in vivo tumor killing.

Example 2: Generation of Monoclonal Antibody to Target Surface Expressed Follicle Stimulating Hormone Receptor (FSHR) and its Engineering to Engage Adaptive Immunity for Targeted Ovarian Cancer Immunotherapy

Despite the important advances in the field of OC therapy, recurrent OC still presents poor prognosis associated with a highly lethal cancer phenotype (Kurnit et al., 2021, Obstet Gynecol. 137 (1): 108-21; Izar et al., 2020, Nat Med. 26 (8): 1271-9; Hamanishi et al., Int Immunol. 28 (7): 339-48). Although there are multiple reasons to suppose that OC would respond favorably to treatment with immunotherapy; yet the immunotherapy response rates among OC patients remain fairly modest (Coleman et al., 2016, Nat Rev Clin Oncol. 13 (2): 71-2). A number of different immune targets have been used in the last few years for targeting OC through active (mesothelin, NY-ESO-1, p53, HER2/Neu, WT-1), passive (H7-B4, EpCAM, CA-125, CD-25, folate receptor a, PD-1/PDL-1, CTLA-4) and adoptive approaches (adoptive T-cell therapy, natural-occurring T-cell therapies, genetically modified T-cell therapies, DC therapies) (Schwab et al., 2014, Immunotherapy. 6 (12): 1279-93). Notably, a prime obstacle in the development of targeted therapies is finding targets with specific expression confined to the surface of tumor cells, but not off target tissues (Perales-Puchalt et al., 2017, Clin Cancer Res. 23 (2): 441-500). FSHR is one such target with selective expression on ovarian granulosa cells (Perales-Puchalt et al., 2017, Clin Cancer Res. 23 (2): 441-500) and thus tools were developed allowing for its consideration as a potential target in OC. Follicle stimulating hormone or FSH is a critical ovarian epithelial cell growth-inducing factor, which functions through binding to FSHR. Overexpression of FSHR is responsible for the upregulation of oncogenic pathways and increased EOC proliferation. Hence FSHR could be utilized as an important therapeutic target for directing T cells against OC (Perales-Puchalt et al., 2017, Clin Cancer Res. 23 (2): 441-500). The potential for tumor impact, tolerability, and safety of targeting FSHR by vaccination was evaluated using an immunocompetent mouse model (Perales-Puchalt et al., 2019, Mol Ther. 27 (2): 314-25). Injection of optimized DNA sequences followed by electroporation confers overexpression of the protein in its native conformation capable of eliciting potent cellular and humoral immune responses (Tebas et al., 2017, N Engl J Med; Yan et al., 2013, Cancer Immunol Res. 1 (3): 179-89). Here this work was extend using this approach for generation of anti-FSHR monoclonal antibodies and study the resulting reagents as novel biologics. The generation and characterization of a potent anti-FSHR antibody and study its application as a tool for immunotherapy of OC is described. Several clones were identified, among them clone D2AP11 which was selected for additional study based on its consistent high cell binding ability in Flow analysis. The MAb supports detection of FSHR expression in samples by multiple methods, including flow cytometry. IFA, and immunohistochemistry. This suggests additional study is warranted for this new reagent to extend its potential use in the clinic where it may aid in determining the FSHR status of patient samples for personalized medicine approaches.

An important recent tool in the field of antibody technology for cancer therapy are bispecific T cell engager approaches (Bhojnagarwala et al., 2022, Molecular Therapy: Oncolytics. S2372-7705 (22): 00092-4). Bispecific T cell engagers can redirect both CD4 as well as CD8 T cells for the killing of tumor cells and are independent of intrinsic antigen specific TCR recognition by the T cells (Dao et al., 2015, Nat Biotechnol. 33 (10): 1079-86). While most of these are in preclinical and early clinical study, there are a very few approved cancer bispecific products. Blincynto (blinatumomab) is used as a therapy for acute lymphoblastic leukemia (ALL) that targets CD19 on B cells and engages T cells through linked anti-CD3 binding (Sheridan, 2021, Nat Biotechnol. 39 (3): 251-4); tebentafusp-tebn (Kimmtrak, Immunocore Limited), a bispecific gp100 peptide-HLA-directed CD3 T cell engager was approved for patients with unresectable or metastatic uveal melanoma (FDA approves tebentafusp-tebn for unresectable or metastatic uveal melanoma. Accessed Apr. 20, 2022. fda.gov) supporting the importance of this approach. Blinatumomab displayed antileukemic function in adults with relapsed/refractory B-precursor ALL depicted by negative prognostic factors. Most reported grade 3 or worse adverse events include febrile neutropenia, neutropenia and anemia. Patients displayed grade 3 cytokine release syndrome (2%) and neurologic events of worst grade 3 (11%) or 4 (2%) (Topp et al., 2015, Lancet Oncol. 16 (1): 57-66). Treatment with tebentafusp led to longer overall survival of metastatic uveal melanoma patients with no prior treatment received. The most frequent treatment-associated adverse events reported were cytokine-mediated events and skin-related events, including rash (83%), pyrexia (76%), and pruritus (69%). However, the incidence and severity of these events were reported to decrease after the first three or four doses (Nathan et al., 2021, N Engl J Med. 385 (13): 1196-206). The benefit of these bispecific T cell engager approaches 304 in severe disease appear of importance and more studies can benefit the patients with severe disease.

Based on the specificity of the D2AP11 anti-FSHR clone, it was reasoned that this could be an important target for OC bispecific T cell engager (TCE) approach. D2AP11-TCE was highly potent in tumor specific cell killing, as evaluated against a panel of different human ovarian tumor cells. Cell lines which display resistance against different chemotherapies including HDAC inhibitors, Wee inhibitors, microtubule stabilizers, DNA alkylating agents etc. (Sakai et al., 2009, Cancer Res. 69 (16): 6381-6) were included. Nearly 23% of OC patients relapse within 6 months after their primary chemotherapy with another 60% of patients reported to relapse after 6 months (Pignata et al., 2017, Ann Oncol. 28 (suppl_8): viii51-viii6). Treatment escapes represent significant hurdles for ovarian cancer therapeutic approaches. D2AP11-TCE showed significant killing against these different drug resistant OC cell lines; OVISE, CaOV3, Kuramochi, OVCAR3, OVCAR4 and PEO-4. Germline mutations in BRCA1 or BRCA2 tumor suppressor genes, which play important roles in homologous recombination are found in approximately 10% of EOC cases (Safra et al., 2011, Mol Cancer Ther. 10 (10): 2000-7; Hennessy et al., 2009, Lancet. 374 (9698): 1371-82). Despite demonstrating impressive activity in case of sporadic high grade serous OC as well as BRCA-related OC, unfortunately, as with classical chemotherapy, many patients are eventually reported to acquire resistance to Poly-ADP-ribose polymerase inhibitors (PARPi) treatment (Yang et al., 2020, Front Immunol. 11:577869; McMullen et al., 2020, Cancers (Basel). 12 (6); Franzese et al., 2019, Cancer Treat Rev. 73:1-9; Liu et al., 2014, Gynecol Oncol. 133 (2): 362-9), supporting the development of approaches targeting such OC tumors remain important. Interestingly, D2AP11-TCE was highly effective against cells harboring mutations in the BRCA1 (OVISE) and BRCA2 (Kuramochi, PEO-4) and also gene signatures exhibiting resistance to PARPi (PEO-4) (Sakai et al., 2009, Cancer Res. 69 (16): 6381-6). Thus, D2AP11-TCE seem to have clinical benefit against a diverse panel of genetic and immune escape that has been previously documented in OC therapy. These studies provide a foundation supporting D2AP11-TCE might have a value in combination with other therapeutic approaches such as, chemotherapy, small molecules, and immunomodulatory drugs such as checkpoint inhibitors. However, future studies addressing this question are important.

Direct DNA in vivo delivery was used to rapidly evaluate this new potential tool in OC challenge models. Notably, the T cell engager targeting FSHR was found to have high potency in attenuating tumor burden/tumor progression in vivo in ovarian tumor bearing mice model, almost eliminating the implanted tumors. PBMCs from 12 different healthy human donors were used across these studies. This is also the first scFV based therapeutic developed for FSHR. This could be useful for designing other scFV based therapeutics such as CART cells targeting FSHR for OC.

These studies demonstrate for the first time the utility of targeting FSHR for a major subset of OC and highly immune potent bispecific tools focused on FSHR and CD3, to impact tumor growth in vivo. Targeting diverse populations of OC and the ability to complement currently available tools for treatment of this disease support study in additional FSHR+tumor models. Further studies are warranted for potential translational advancement of this new tool for a variety of ovarian and cancers expressing FSHR.

The methods used in the experiments are now described

### Cell Lines and Animals

Cell lines used in the studies include ID8-Defb29/Vegf-a-Fshr, ID8-Defb29/Vegf-a, OVCAR3, CaOV3, and TOV-21G, OVISE, OVCAR4, PEO-4, Kuramochi cells, Human embryonic kidney 293T, Expi293F, gastric adenocarcinoma AGS and murine myeloma cell line Sp2.0/0. OVCAR3, OVISE and Kuramochi cells were retrovirally transduced with human FSHR as described previously (Perales-Puchalt et al., 2019, Mol Ther. 27 (2): 314-25). K562 and A20 were purchased from ATCC and retrovirally transduced to express human and murine FSHR respectively. The expression vector pBMN-I-GFP was purchased from Addgene.

### Design of FSHRxCD3 TCE

A FSHRxCD3 DNA encoded bispecific T cell engager (TCE) was designed by encoding a codon optimized scFv of FSHR MAb (D2AP11) followed by the scFv of a modified UCHT1 anti-human CD3 antibody with the addition of an IgE leader sequence. The construct was subcloned into a modified pVax1 expression vector (Perales-Puchalt et al., 2019, JCI Insight. 4 (8)). FSHRxCD3 TCE is designated as D2AP11-TCE.

### Flow Cytometry

A BD LSRII flow cytometer for was used staining of cells. BD FACS Aria cell sorter (BD Biosciences) was used for the sorting of FSHR stably expressing cells. Anti-human antibodies used were directly fluorochrome conjugated. PE-secondary anti-human (H+L) (Invitrogen), PE/AF647-secondary anti-human F(ab′)2 (Jackson ImmunoResearch Laboratories Inc) and APC secondary anti-mouse IgG (BioLegend) were used. Live/Dead Violet viability kit (Invitrogen) was used to exclude dead cells from analysis.

### Enzyme-Linked Immunosorbent Assay (ELISA)

For isotyping D2AP11, the ELISA plates were coated with D2AP11 in PBS overnight. Following, the plate was blocked and the following HRP conjugated antibodies were added: anti-mouse IgA, anti-mouse IgM, anti-mouse IgG1, anti-mouse IgG2a, anti-mouse IgG2b, 392 anti-mouse IgG3, anti-mouse kappa light chain (all from Bethyl).

### Cyclic AMP Determination

25,000 K562 or K562-hFSHR cells were plated in 96 well plates. Cells were washed twice with warm PBS then resuspended in 100 μl of serum-free RPMI with 0.5 mM IBMX (Cayman chemicals) with or without D2AP11 antibody. After incubation for 30 min at 37° C. FSH (50 ng/ml or 1 μg/ml) or PBS was added. One hour later the cells were washed with ice cold PBS, lysed them and performed cyclic AMP determination according to the manufacturer's instructions (Cell Signaling).

### Immunoblotting

Protein extraction, denaturation and Western blotting were performed as previously described (Bordoloi et al., 2021, ACS Pharmacol Transl Sci. 4 (4): 1349-61; Tesone et al., 2016, Cell Rep. 14 (7): 1774-86; Bordoloi et al., 2021, Genes Cancer. 12:51-64). Membranes were blotted with: anti-Phospho-p44/42 MAPK (Erk1/2) (Thr202/Tyr204) (#9101, Cell Signaling), and anti-p44/42 MAPK (Erk1/2) (clone 137F5, Cell Signaling). Images were captured with ImageQuantLAS 4000 (GE Healthcare Life Sciences). For detection of D2AP11-TCE expression, Rabbit anti-human F(ab′)2 (Jackson ImmunoResearch Laboratories Inc.) and Goat-anti Rabbit secondary antibody (LiCOR) were used and membranes were scanned using a LI-COR Odyssey CLx imager.

In vitro cytotoxicity analysis through measurement of luciferase expression 10,000 OVCAR3 cells per well in a 96-well plate were plated and after 18 hours primary peripheral blood mononuclear cells (PBMC) were added. After a 4 h co-incubation, the cells were stained with 7AAD (Invitrogen), Annexin V (Biolegend) and anti-human CD45 (Biolegend) then a flow cytometry-based cytotoxicity assay was performed as described previously (Perales-Puchalt et al., 2017, Clin Cancer Res. 23 (2): 441-500). K562 and K562-FSHR with firefly luciferase were stably transfected. 20,000 K562 and K562-FSHR expressing luciferase were plated in a 96-well plate and co-incubated for 5 hours with PBMC. Following the incubation, the cells were lysed and luciferase expression was measured using CytoTox Glo (Promega) as previously described (Zhang et al., 2009, Cancer Res. 69 (16): 6506-14). Cytotoxicity was calculated as (maximum viability control-individual well)/(maximum viability control-maximum death control)*100 as a percentage or relative to the control (PBMC with mouse IgG2a isotype control C1.18.4).

In Vitro Cytotoxicity Analysis Using xCELLigence Real Time Cell Analyzer

In vitro cytotoxicity assay was performed based on impedance using xCELLigence real time cell analyzer equipment (RTCA), Agilent Technologies, USA. The impedance is expressed as arbitrary unit called cell index (Curdagi et al., 2021, Mol Ther). Target cells were seeded into disposable sterile 96-well E-plates of the xCELLigence RTCA device at final cell concentration of 1×10⁴-2×10⁴ cells per well. The instrument has been placed in a CO₂ incubator during the experiment and controlled by a cable connected to the control unit. The 96-well E-Plate was placed in the xCELLigence RTCA device and incubated for 18-24 hours. Subsequently, the effector cells (Human PBMCs; E (Effector): T (Target) ratio=5:1/10:1) and treatments were added. Real time analysis was performed for 3-7 days. The electrical conductivity is converted into the unitless cell index (CI) parameter by the xCELLigence device in every 15 minutes and images were captured at the 1-hour intervals. The data generated are normalized as per the time point when the effector cells and TCE were added to the target cells and were analyzed using RTCA/RTCA Pro Software. Human PBMCs and T cells from healthy donors were provided by the Human Immunology Core of the University of Pennsylvania.

### Immunohistochemistry and Immunocytochemistry

Mouse tumors were frozen in OCT (TissueTek) and frozen sections cut. HEK293T cells were grown on top of poly-L-lysine-coated cover slides (Sigma) and transfected using a human or murine FSHR expression vector. Slides were then 438 fixed with 4% paraformaldehyde and permeabilized with 0.5% Triton X-100 in PBS. Sections were blocked using 5% normal goat serum followed by staining with D2AP11 antibody then AF647-conjugated secondary antibodies specific for human or mouse IgG (Invitrogen). Slides were viewed using Leica TCS SP-5 confocal microscope and Leica LAS-X software (immunocytochemistry) or Nikon ECLIPSE 80i microscope and the NIS-Element Imaging (immunohistochemistry). For Immunohistochemistry analysis of TMA slides (US Biomax), they were de paraffinized, rehydrated, followed by antigen retrieval, blocking with 5% normal goat serum, staining with D2AP11 Antibody and then biotinylated anti-mouse secondary antibody (Vector Laboratories). Then the TMA slides were incubated with peroxidase solution (Vector Laboratories) followed by DAB substrate and countersting with hematoxylin (Leica). The slides were viewed and imaged using Nikon NIS Element Imaging system (20×, scale: 500 μm).

### Cytokine Secretion Profile Analysis

OVCAR3-FSHR (Target) cells were plated at the density of 1×104 cells/well. After overnight incubation, PBMCs (Effector cells: E:T=5:1) and treatments (D2AP11-TCE and pVax1) were added to the target cells. Post 48 hours, the supernatants were collected and analyzed for secreted cytokines by LEGENDplex™ Human CD8/NK Panel multiplex bead-based assay (Biolegend) as per manufacturer's protocol.

### Tumor Challenges

NOD/SCID-γ (NSG) mice were challenged with K562 and K562-FSHR cells. NSG mice were injected with 4×106 K562 or K562-FSHR cells on the right flank subcutaneously. 7 days after, when the tumor became palpable, mice were inoculated with pVax1 (100 μg), or D2AP11-TCE (100 μg). The same day when expression vector was given, 4×10⁶ human T cells were injected intraperitoneally into each mouse. The mice were inoculated with DNA twice, one week apart. For OVCAR3-FSHR challenged mice model, mice were inoculated with OVCAR3-FSHR cells on the right flank subcutaneously. 14 days after when the tumor became palpable, mice were inoculated with pVax1 (100 μg), or D2AP11-TCE (100 μg). The same day, 10×10⁶ human T cells were injected intraperitoneally into each mouse. The mice were inoculated twice, two weeks apart. Tumor sizes were monitored periodically via caliper measurements. Mice were euthanized upon developing signs of graft versus host disease (GVHD). Tumor volume (V) was calculated as per the formula V=[(length Xwidth²)]/2; width is the side with smaller measurement. Animal experiments were approved by the Institutional Animal Care and Use Committee at The Wistar Institute. Human T cells from healthy donors were provided by the Human Immunology Core of the University of Pennsylvania.

### Statistical Analysis

All statistical analyses were done using Graph Pad Prism. A p-value<0.05 was considered statistically significant. Differences between the means of experimental groups were calculated using a two-tailed unpaired Student's t test or one-way ANOVA where more than two quantitative variables were measured. Error bars represent standard error of the mean. Comparisons between tumor size at each time point were done using two-way ANOVA with Fisher's least significant difference (LSD) test.

### The Experimental Results are Now Described

### Generation and Flow Cytometry Screening of Anti-Human FSHR Antibodies

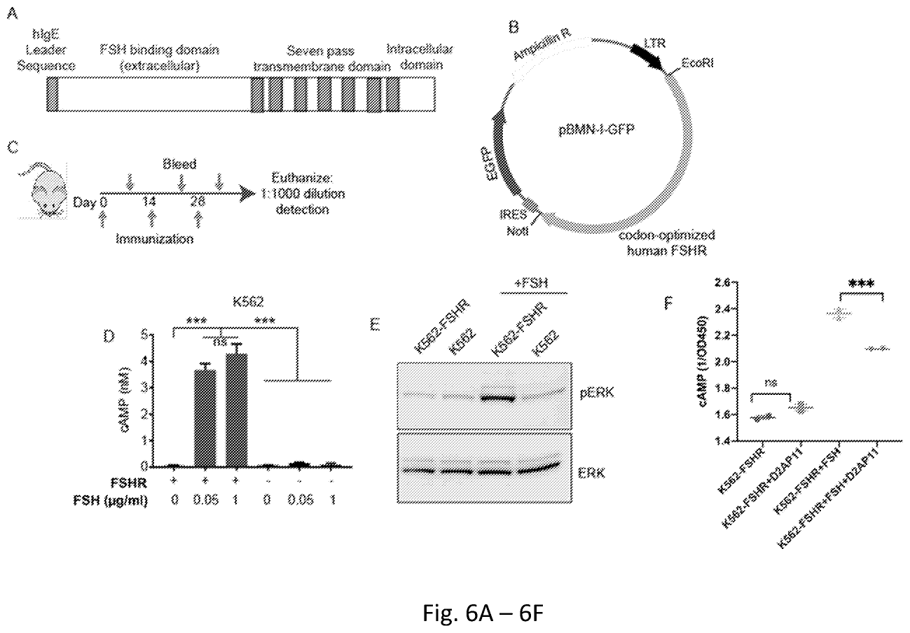

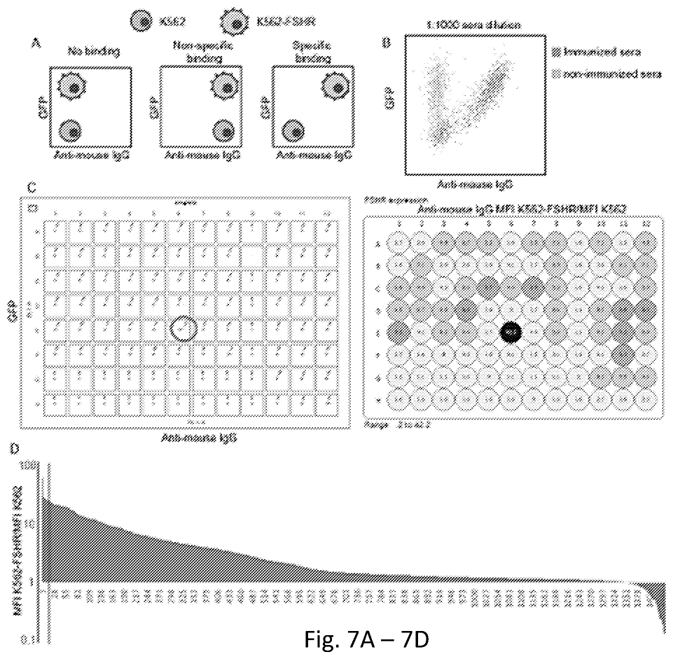

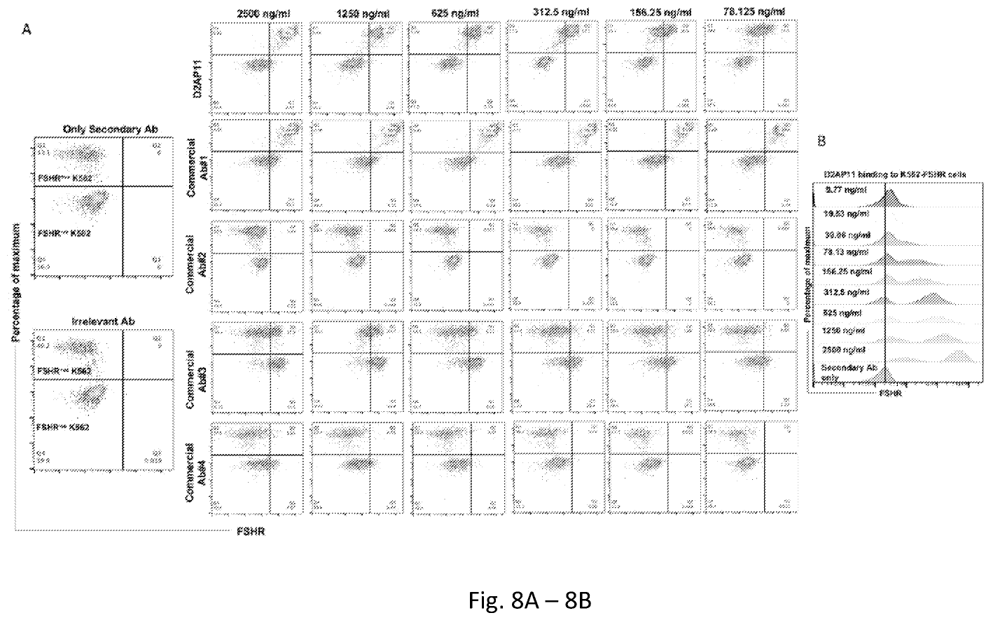

FSHR is a tumor-associated antigen present in ovarian cancer (Perales-Puchalt et al., 2017, Clin Cancer Res. 23 (2): 441-500), prostate cancer (Mariani et al., 2006, J Urol. 175 (6): 2072-7), and the neovessels of 80% of cancers (Radu et al., 2010, N Engl J Med. 363 (17): 1621-30). It is a G-coupled protein receptor with seven transmembrane domains (Fan et al., 2005, Nature. 433 (7023): 269-77). This complex structure presents challenges for classical antigen protein approaches. A codon optimized sequence of the human FSHR (FIG. 6A) was generated for direct in vivo immunization allowing for the generation of responses against a putative native antigen structure on the surface. The FSHR cDNA was subcloned into a characterized expression vector (FIG. 6B) and inoculated mice for the generation of antibody responses using direct plasmid injection followed by in vivo electroporation. Animals were immunized biweekly, and sera were collected a week after each immunization for analysis of antibody levels (FIG. 6C). To detect anti-FSHR antibodies that would bind to native FSHR expressed in the cell membrane, K562 cells were stably transduced to overexpress human FSHR (K562-FSHR). To validate the correct folding and functionality of the recombinant FSHR, the response of K562-FSHR cells to follicle stimulating hormone (FSH) was tested. As expected, K562-FSHR increased their production of cyclic AMP and ERK phosphorylation upon FSH stimulation, but no response was observed in the parental K562 (FIG. 6D&E). In a preliminary experiment, it was observed that the anti-FSHR antibody D2AP11 partially blocked cAMP production by FSH stimulation of FSHR. No difference was seen in cAMP production in K562-FSHR cells only in the presence of D2AP11 antibody without addition of FSH (FIG. 6F), suggesting additional studies in this area are important to understand the mechanism and to further confirm this observation (Zhu et al., 2018, Trends Endocrinol Metab. 29 (8): 571-8024; Haldar et al., 2022, Int J Biol Sci. 18 (2): 675-92). To monitor the ability of immune sera to bind FSHR, K562 (GFP-) and K562-FSHR (GFP+) cells were combined at equal ratios and sera diluted up to 1:1000 was added, followed by anti-mouse IgG APC conjugated secondary antibody (FIG. 7A&B) (Sakai et al., 2009, Cancer Res. 69 (16): 6381-6) and the fold mean fluorescent intensity (MFI) of K562-FSHR was determined compared to wildtype K562. When a 1:1000 serum dilution exceeded 20-fold MFI, the last immunization was performed 3 weeks from the previous immunization by boosting with FSHR-overexpressing A20 cells. Boosted animals were sacrificed 4 days later for hybridoma generation as described (Choi et al., 2020, Hum Vaccin Immunother. 16 (4): 907-18; Bordoloi et al., 2021, ACS Pharmacol Transl Sci. 4 (4): 1349-61). Two weeks following the fusion, supernatants from fifteen, 96 well plates were screened using flow cytometry to analyze the potential hybridomas (FIG. 7C). Fold-MFI values ranged from 0.2 to 42.2. The top 20 clones based on fold-MFI were expanded for further analysis (FIG. 7D). A highly potent clone, D2AP11 (fold-MFI 42.2) was down selected based on high binding specificity. Next the binding potential of this potent down selected antibody was compared with 4 different commercially available mouse anti-human FSHR antibodies at different concentrations ranging from 2500 ng/ml to 9.77 ng/ml. As shown in FIG. 8A, D2AP11 exhibited high specific binding (K562-FSHR cells) and no nonspecific binding (K562 cells). Commercial Ab #1 showed high binding to K562-FSHR cells, however at concentrations: 2500, 1250 and 625 ng/ml, it showed nonspecific binding to FSHR non expressing wild type K562 cells. Commercial Ab #3 showed modest binding at 2500 and 1250 ng/ml, however there was high nonspecific binding to K562 cells. FSHR binding was not observed in case of commercial Ab #2 and Commercial Ab #4 when evaluated in different concentrations; highest up to 2500 ng/ml. Notably, D2AP11 mAb showed potent binding to concentrations as low as 9.77 ng/ml. The dose dependent binding of this potent mAb clone is shown in FIG. 8B and represents a new potent tool for human FSHR study.

Anti-FSHR Antibody Binds FSHR with High Specificity

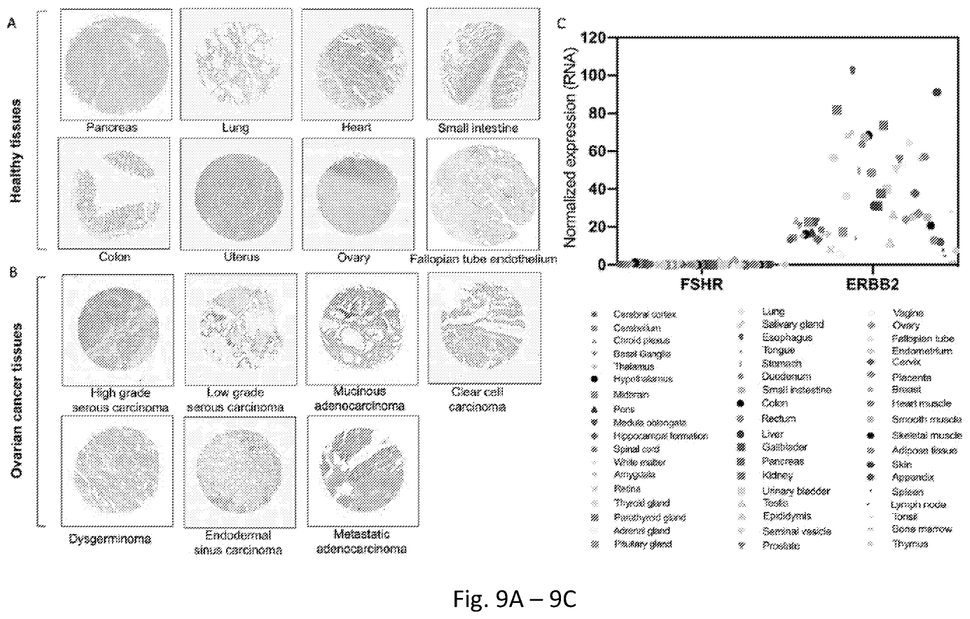

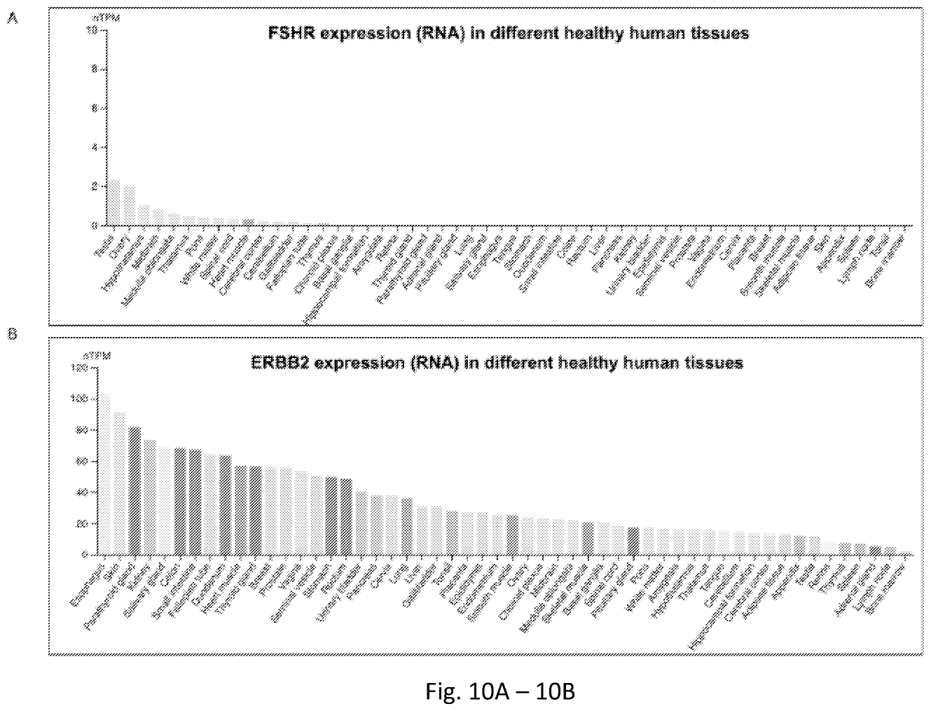

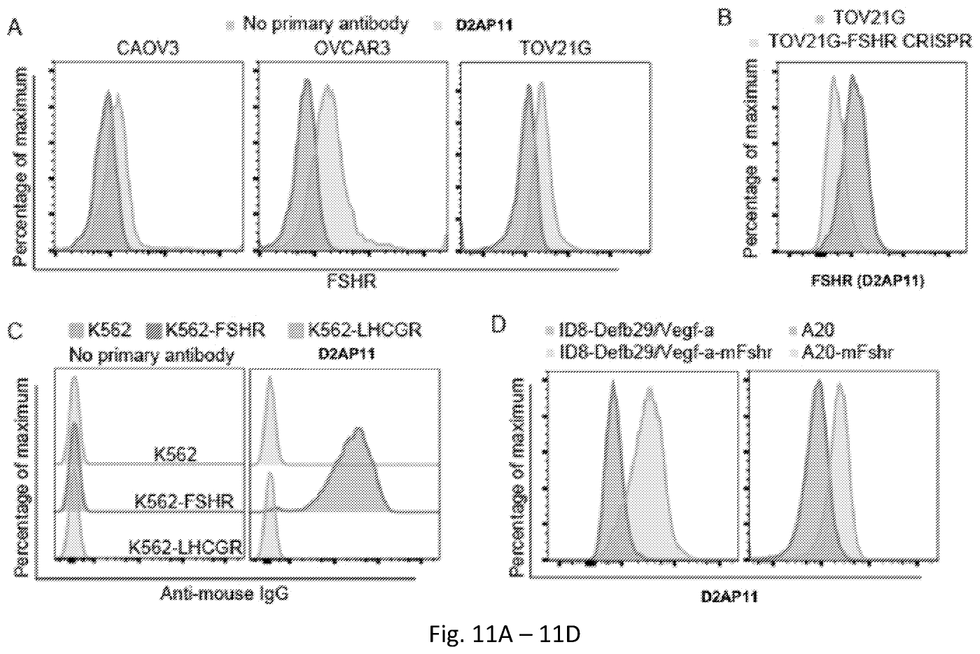

D2AP11 anti-FSHR antibody was characterized in detail for specificity by staining a panel of different healthy human tissues which include pancreas, lung, heart, small intestine, colon, uterus, ovary and fallopian tube endothelium. For most of the healthy tissues, no significant binding of D2AP11 was observed. D2AP11 binding has been seen in ovary and fallopian tube endothelium and high binding in different ovarian cancer tissues (high grade serous carcinoma, low grade serous carcinoma, clear cell carcinoma, dysgerminoma, mucinous carcinoma, endodermal sinus carcinoma and metastatic adenocarcinoma) (FIGS. 9A & B). Further studies on additional healthy and ovarian cancer tissues are important. Based on ‘The Human Protein atlas’ data, the differential expression (RNA) of FSHR was compared with ERBB2/Her2, targeting which monoclonal antibodies and small molecule inhibitors were developed and currently clinical trials focusing on Her2 targeted bispecific immune cell engagers are ongoing (FDA approves tebentafusp-tebn for unresectable or metastatic uveal melanoma. Accessed Apr. 20, 2022. fda.gov; proteinatlas.org). These comparisons on 55 different human tissue types (proteinatlas.org) likely support FSHR deserves additional study for targeted ovarian cancer immunotherapy (FIG. 9C, FIG. 10). D2AP11 was then further characterized in detail for binding to different FSHR expressing cells. D2AP11 binds to ovarian cancer cell lines spontaneously expressing FSHR (Perales-Puchalt et al., 2017, Clin Cancer Res. 23 (2): 441-500; Zhang et al., 2009, Cancer Res. 69 (16): 6506-14). Cell lines (CAOV3, OVCAR3 and TOV-21G) all showed the expected expression of FSHR by D2AP11 staining (FIG. 11A). To further confirm that the signal elicited by D2AP11 corresponded to FSHR, CRISPR mediated deletion of FSHR in the TOV-21G cell line was performed. Flow cytometric staining with D2AP11 antibody showed absence of binding to TOV-21G cell line after CRISPR mediated FSHR knock-out (FIG. 11B). This clone was highly specific for FSHR as K562 cells transfected with LHCGR, the homologous protein to FSHR (sequence homology ˜46% in the ECD and ˜72% in the 7TMD) (Ulloa-Aguirre et al., 2018, Front Endocrinol (Lausanne). 9:707) did not show cross-reactivity by flow cytometry analysis (FIG. 11C). It was further tested if D2AP11 was also able to bind murine FSHR, as mouse models of disease are important to study the in vivo efficacy and safety of new therapies. Murine FSHR was expressed in mouse tumor lines A20 and ID8-Defb29/Vegf-a and again tested binding of D2AP11 to transfected and untransfected cells by flow cytometry. It was found that D2AP11 successfully bound murine FSHR as it did human FSHR (FIG. 11D).

### Anti-FSHR Antibody for Detection of FSHR+Tumor Cells

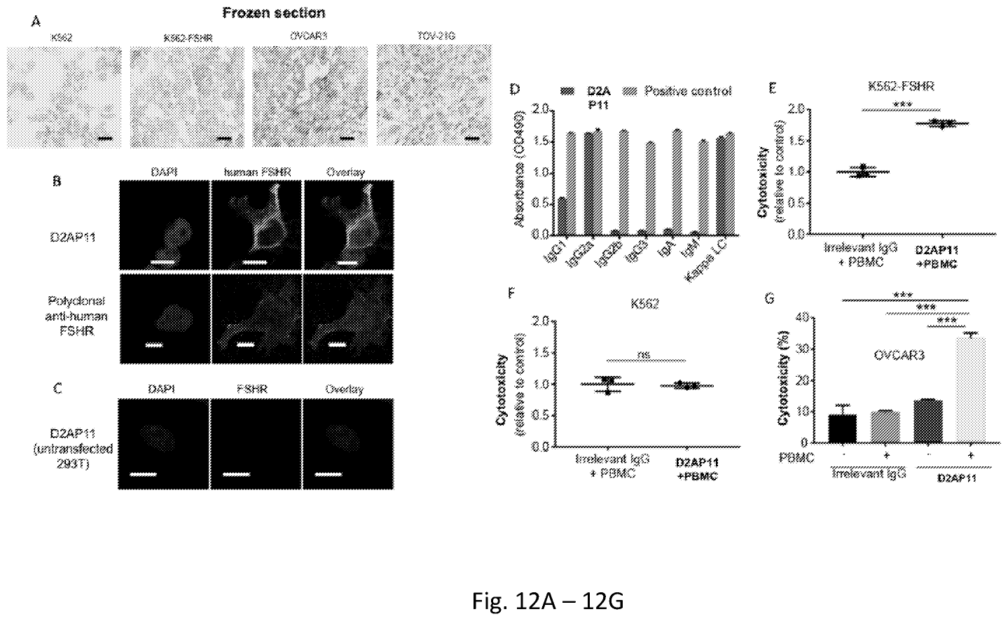

Immunohistochemical detection of proteins from biological samples is a common way of determining protein expression from tumors or other specimens to better classify them for prognostic or therapeutic purposes. To further explore if D2AP11 detects FSHR+tumor cells in immunohistochemistry, solid tumors in NSG immunodeficient mice were generated. To generate tumors, 5 million K562, K562-FSHR, OVCAR-3 or TOV-21G were injected in 50% PBS/Matrigel (Corning) into the axillary flank of NSG mice. D2AP11 detected FHSR from frozen tumor sections (FIG. 12A). Human FSHR-transduced 293T cells were stained with D2AP11. D2AP11 was able to bind human FSHR similar to polyclonal anti-human, but not to mock transfected 293T cells confirming this activity (FIG. 12B-C).

### Anti-FSHR Antibody Induces Antibody-Dependent Cell Mediated Cytotoxicity (ADCC)

To determine the isotype of D2AP11, an ELISA was performed and D2AP11 was found to be IgG2a (FIG. 12D), an isotype that can elicit ADCC (Akiyama et al., 1984, Cancer Res. 44 (11): 5127-31). The ADCC capacity was first tested with K562 with or without FSHR. D2AP11 was able to increase the cytotoxic activity of PBMC against K562-FSHR but not against K562 (FIG. 12E&F). To determine its ability to induce ADCC against unmodified FSHR+ovarian cancer cell lines, OVCAR3 cells were cocultured with PBMC in the presence of D2AP11 or an irrelevant IgG2a antibody. It was found that the physiological expression levels of FSHR in the ovarian cancer cells were sufficient to be targeted by D2AP11 mediated cytotoxicity (FIG. 12G) particularly with increasing doses of antibodies.

### Generation, Expression, and Binding of FSHR Targeted Novel Bispecific T Cell Engager

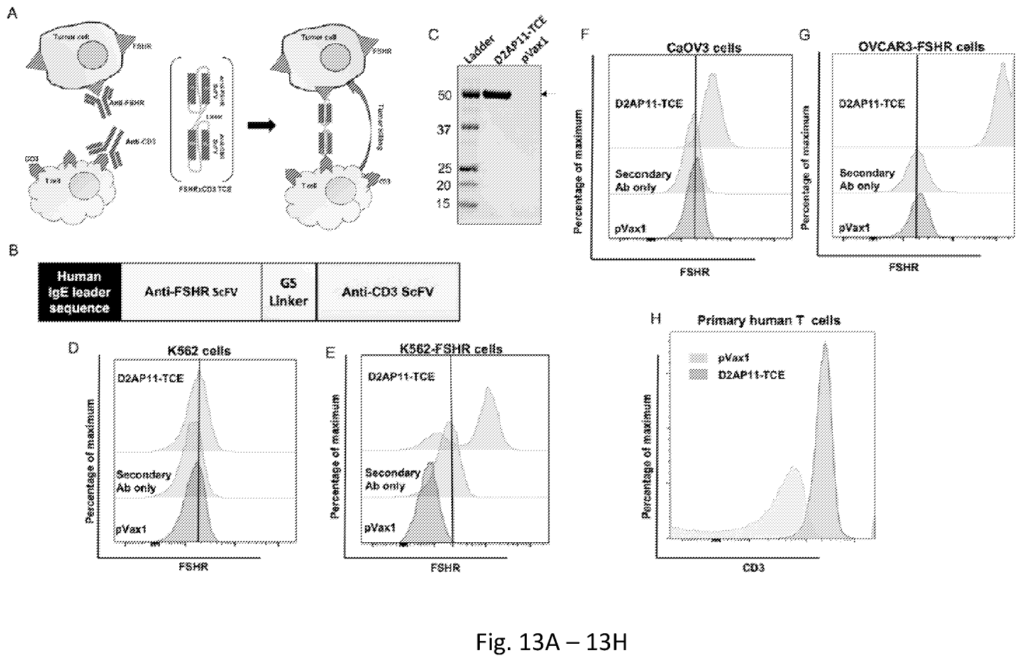

Bispecific T cell engagers represent a recent significant development in the field of monoclonal technology. As D2AP11 anti-FSHR antibody exhibited initial levels of ADCC, it was sought to improve on this potential. A FSHR targeting TCE (D2AP11-TCE) was designed (Gary et al., 2021, iScience. 24 (7): 102699; Patel et al., 2018, Cell Rep. 25 (7): 1982-93 e4; Perales-Puchalt et al., 2019, JCI Insight. 4 (8)). The scFv of the FSHR mAb was genetically optimized and fused with the scFv of an optimized sequence wthat was developed encoding anti-CD3 (modified from UCHT1) (FIGS. 13A & B). D2AP11-TCE was efficiently expressed in vitro upon transfection of the DNA in Expi293F cells (FIG. 13C). This novel bispecific showed no nonspecific binding to K562 cells which do not have natural FSHR expression (FIG. 13D) and retained binding to K562-FSHR cells (FIG. 13E). Binding to FSHR was further confirmed in CaOV3 (FIG. 13F) and OVCAR3-FSHR cells (FIG. 13G). CD3 binding of D2AP11-TCE bispecific was confirmed using primary human T cells (FIG. 13H).

### D2AP11-TCE Induced Potent Killing in Multiple Ovarian Tumor Lines

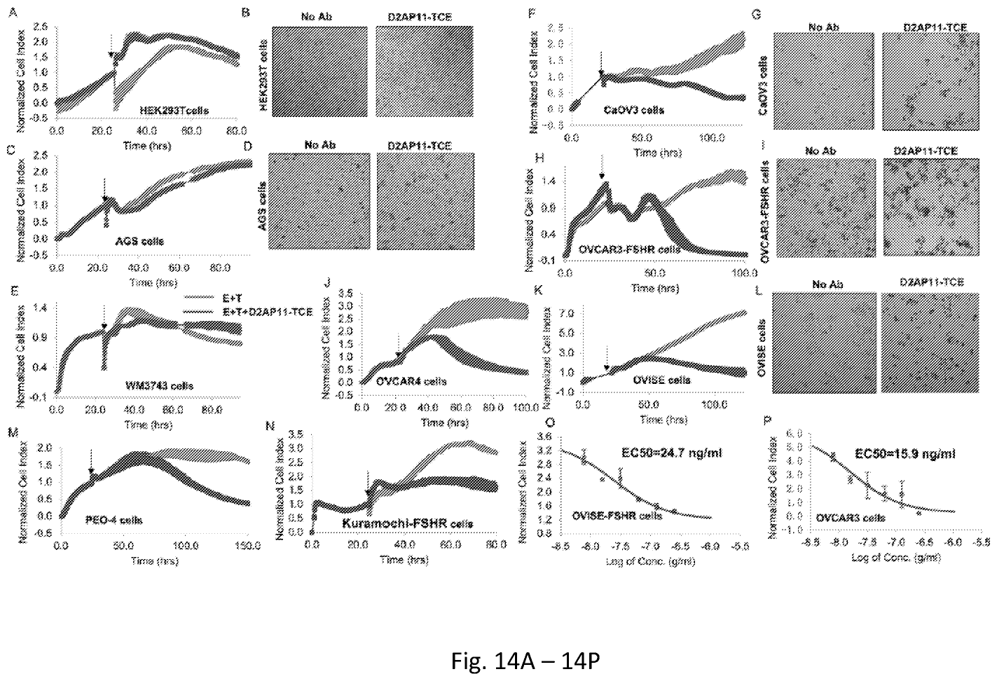

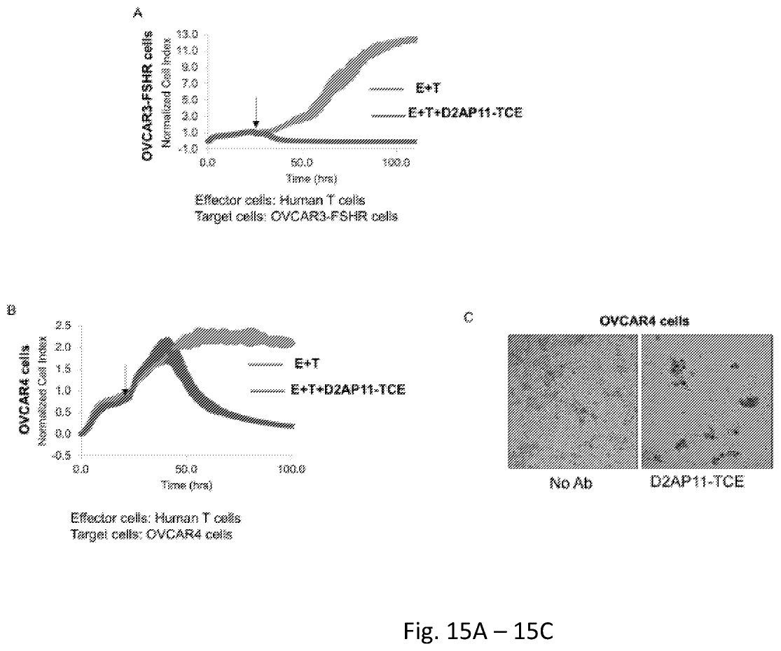

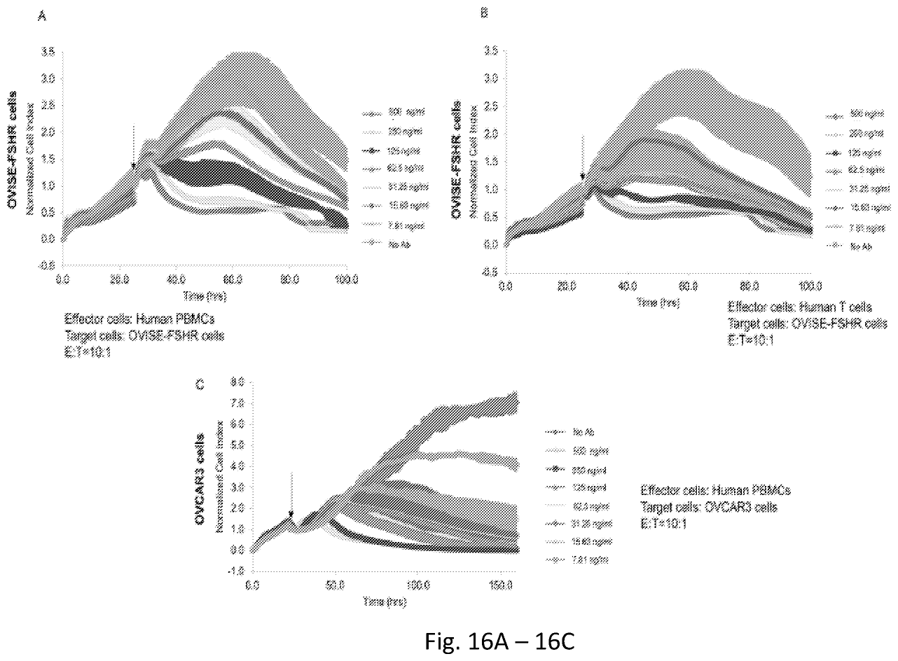

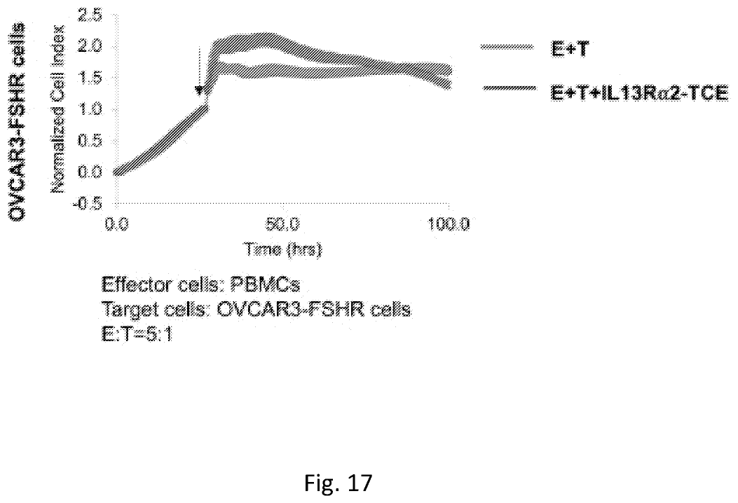

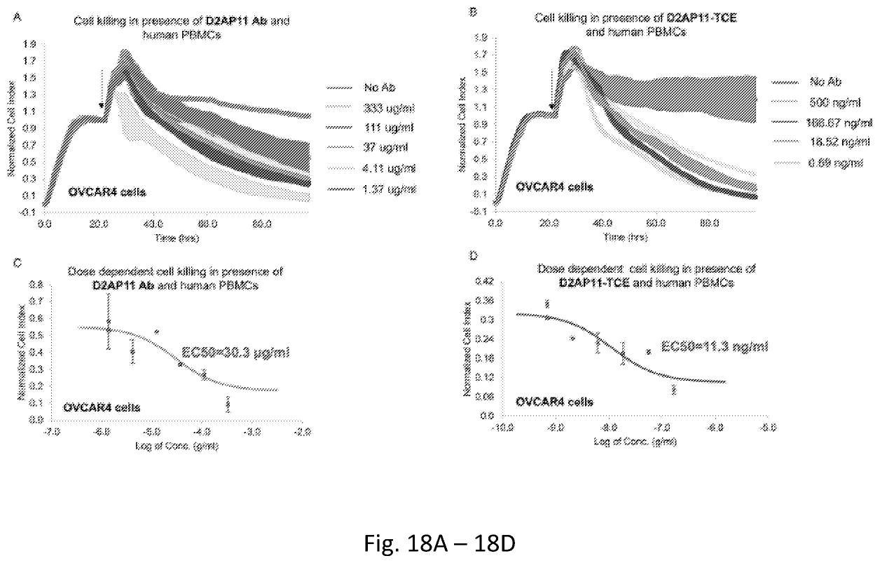

To determine the ability of the FSHR targeted novel bispecific T cell engager; D2AP11-TCE to induce cytotoxicity through activation of T cells, in vitro cytotoxicity assay was performed based on impedance using xCELLigence real time cell analyzer. The target cells (OVISE, CaOV3, OVCAR3-FSHR, OVCAR4, PEO-4 and Kuramochi-FSHR) were placed in the xCELLigence RTCA device, incubated for 18-24 hours, and subsequently, human PBMCs and D2AP11-TCE was added. HEK 293T cells were used as a control; a FSHR-negative cell line (Urbanska et al., 2015, Cancer Immunol Res. 3 (10): 1130-7). Notably, off target killing against FSHR-negative HEK293T cells was not observed (FIG. 14A&B). As additional controls, two other FSHR non expressing cells were used such as AGS gastric adenocarcinoma (FIG. 14C&D) and WM3743 (FIG. 14E) human melanoma cells and D2AP11-TCE did not induce off target toxicities in those two cells as well. Evaluation of different FSHR expressing ovarian tumor cells demonstrated that D2AP11-TCE was highly efficient in the killing of CAOV3 (FIG. 14F&G), OVCAR3-FSHR (FIG. 14H&I, FIG. 15A), OVCAR4 (FIG. 14J, FIG. 15B&C), OVISE (FIG. 14K&L), PEO-4 cells (FIG. 14M) and Kuramochi-FSHR cells (FIG. 14N). Dose dependent killing was observed in OVISE-FSHR and OVCAR3 cells in the presence of D2AP11-TCE and human PBMCs/T cells with EC50 value of 24.7 ng/ml and 15.9 ng/ml respectively (FIG. 14O&P, FIG. 16). OVCAR4, a high grade serous ovarian adenocarcinoma cell line is reported to have distinct positive expression of the surface receptor; FSHR (depmap.org) and D2AP11-TCE induced potent killing in this cell line in the presence of human PBMCs as well as human T cells (FIG. 14J, FIG. 15B&C). The studied ovarian tumor lines harbor different cancer driver mutations and exhibit resistance to multiple anticancer drugs (Table 1). Of note, Kuramochi and PEO-4 bear BRCA2 mutations and the later also exhibits resistance to PARP inhibitors (Sakai et al., 2009, Cancer Res. 69 (16): 6381-6, He et al., 2018, Nature. 563 (7732): 522-6). In this assays there is no escape from D2AP11-TCE killing in those two resistant ovarian tumor lines as well. As shown in the images (FIG. 14D, F &H), post 3 days addition of effector cells and treatment with D2AP11-TCE, no attached tumor cells were observed in the treated wells. However, in control HEK293T cells (FIG. 14B), all cells were found to be growing healthy post 2-3 days addition of effector cells and D2AP11-TCE. Additionally, an irrelevant TCE targeting human IL-13 receptor alpha 2 (IL13Rα2-TCE) did not exhibit toxicity in OVCAR3-FSHR cells in the presence of human PBMCs, indicating the role of D2AP11 arm for specific killing of FSHR expressed tumors (FIG. 17). Of note, D2AP11-TCE was able to induce significant toxicity to FSHR positive OVCAR4 cells when compared to D2AP11 antibody, at concentrations around 1000-fold lower indicating the enhanced killing efficacy of D2AP11 through its design as bispecific engager; D2AP11-TCE (FIG. 18A&B). EC50 values of D2AP11 and D2AP11-TCE were obtained at 30.3 μg/ml and 11.3 ng/ml respectively, indicating ˜ 1000-fold higher potency of D2AP11-TCE compared to the anti-FSHR ab: D2AP11 (FIG. 18C&D).

TABLE 1
Different ovarian tumor lines used in the study, types,
cancer driver mutations, and drug resistance

| Name | Type | Cancer driver mutations | Resistant drugs (Drug targets) |
|---|---|---|---|
| OVISE | Ovarian clear cell | ARID1A, ARID1B, BRCA1, CALR, | FEN1_3940(FEN1), Tanespimycin |
| | adenocarcinoma | ARHGAP35, SDHA, DDX5, ETV4, | (HSP90) |
| | | HSP90AB1, NFKBIE, FGFR4, LYL1, | |
| | | MDM2, PIK3CA, PPP2R1A, CD79B, | |
| | | PRKACA, PTPRB, RGL3, STAT3, | |
| | | UGT2B17, WNK4, STAT5B, TGIF1, | |
| | | ZNF429, ZNF780A | |
| CaOV3 | High grade ovarian | ASPSCR1, HSP90AB1, AMER1, ATR, | Dacinostat (HDAC) |
| | serous | BCL6, BCORL1, CAMTA1, BCR, CBLB, | |
| | adenocarcinoma | CCND2, CHD4, CHEK2, COL1A1, CR1, | |
| | | CRLF2, DGCR8, EIF4A2, ERC1, ETV5, | |
| | | FGFR3, HLAB, KLHL6, KMT2A, LPP, | |
| | | MAPK1, MB21D2, MN1, NFKBIE, | |
| | | TBL1XR1, NSD2, NT5C3A, P2RY8, | |
| | | PAK2, PDPR, PIK3CB, PMS2, POLQ, | |
| | | POU5F1, PTK6, PTPN6, RASA2, | |
| | | RSPH10B2, SDHA, SFRP4, SMARCB1, | |
| | | CTLCL1, SOX2, SPOP, STK11, SUSD2, | |
| | | TERT, TP53, TP63, ZBTB20, ZNF-384, | |
| | | CCND3, MAP3K13, ZNF148, | |
| Kuramochi | High grade ovarian | ABI1, AKT2, ARHGAP35, BCL3, | Dactolisib (PI3K -class 1, MTORC1, |
| | serous | BRCA2, CBLC, CCNE1, CD79A, CDH10, | MTORC2), Wee1 inhibitor (WEE1, |
| | adenocarcinoma | CEBP1, CHCHD7, CIC, DEK, EIF3E, | CHEK1), Buparlisib (PI3Kalpha, |
| | | ERCC2, EXT1, FAM135B, GNAS, H4C9, | PI3Kdelta, PI3Kbeta, PI3Kgamma), UMI- |
| | | HEY1, HLA-B, IL7R, KMT2B, KMT2C, | 77 (MCL1), Docetaxel (Microtubule |
| | | KRAS, LATS2, LIFR, MYC, NBN, | stabilizer), AZD5438 (CDK2), |
| | | NCOA2, NDRG1, NEKBIA, NFATC2, | Gallibiscoquinazole, |
| | | NIPBL, PLAG1, POU2F2, PREX2, | Mitoxantrone, Irinotecan (TOP1), |
| | | PRKD2, PTK6, RAD21, RECQL4, | Entinostat (HDAC1, HDAC3), Oxaliplatin |
| | | RHPN2, RSPH10B2, SALL4, SDC4, | (DNA alkylating agent) |
| | | SDHA, SS18L1, TERT, TP53, UBR5, | |
| | | UGT2B17, ZMYM2 | |
| OVCAR3 | High grade ovarian | AKT2, CAMTA1, CASZ1, CEBPA, | Sepantronium bromide (BIRC5), Foretinib |
| | serous | CN0T3, DDX5, EFTUD2, ELK4, EPHA2, | (MET, KDR, TIE2, VEGFR3/FLT4, RON, |
| | adenocarcinoma | ETV4, KDM5C, KMT2B, MTOR, | PDGFR, FGFR1, EGFR), Belinostat |
| | | NPEPPS, PCM1CCNE1, PRDM16, | (HDAC1), CAY10603 (HDAC1, HDAC6), |
| | | PRDM2, RHPN2, RPL22, SLC45A3, | AR-42 (HDAC1), CUDC-101 (HDAC1-10, |
| | | SMC1A, SPEN, TNFRSF14, TP53, | EGFR, ERBB2), JW-7-24-1 (LCK), |
| | | ZCRB1, ZNF331, ZNF721, ZNF780A, | Omipalisib (PI3K-class 1, MTORC1, |
| | | ZXDB | MTORC2), QL-XII-47 (BTK, BMX), THZ- |
| | | | 2-102-1 (CDK7), AT-7519 (CDK1, CDK2, |
| | | | CDK4, CDK6, CDK9), UNC0638 (G9a and |
| | | | GLP methyltransferases), QL-X-138 (BTK), |
| | | | LDN-193189 (BMP), KIN001-244 (PDK1), |
| | | | OSI-027 (MTORC1, MTORC2), WZ3105 |
| | | | (SRC, ROCK2, NTRK2, FLT3, IRAK1, |
| | | | others), WZ3105 (SRC,ROCK2, NTRK2, |
| | | | FLT3, IRAK1, others) |
| OVCAR4 | High Grade Ovarian | BRAF, BREB3L2, CAMTA1, CCNE1, | Pictilisib (PI3K-class 1), Eg5_9814 |
| | Serous | CEBPA, CNOT3, CUX1, EZH2, | (KSP11), Taselisib (PI3K- beta sparing), |
| | Adenocarcinoma | FAM135B, KEL, KLK2, KMT2C, MET, | Mirin (MRE11), AZD2014 (mTORC1, |
| | | MYC, NDRG1, PEG3, POLD1, POT1, | mTORC2), AZ960 (JAK2, JAK3), |
| | | PPP2R1A, RECQL4, RHPN2, SMO, | Teniposide, CDK9_5576 (CDK9), |
| | | SND1, TP53, TRIM24, TRRAP, U2AF2, | AZD5363 (AKT1, AKT2, AKT3, ROCK2), |
| | | UGT2B17, ZNF331 | CDK9 5038 (CDK9) |
| PEO-4 | Ovarian | BRCA2 | Cisplatin (DNA alkylating agent), PARP |
| | cystadenocarcinoma | | inhibitor (PARP) |

### Cytokine Secretion Profile of Novel FSHR Targeting T Cell Engager

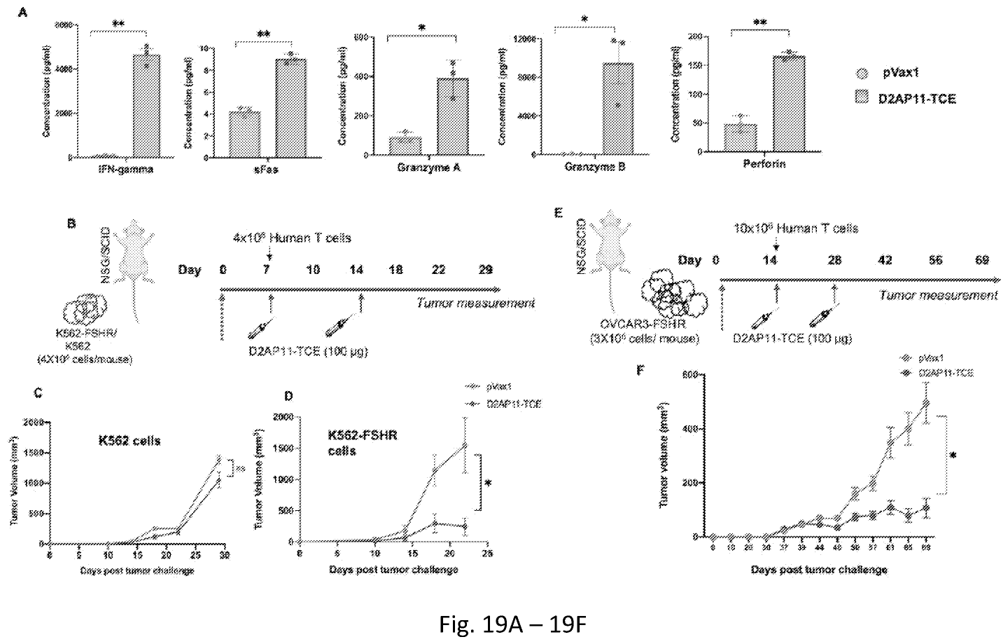

Cytokines are involved in promoting the proliferation, survival, differentiation, and activation of lymphocytes (Romain et al., 2014, Blood, 124 (22): 3241-9). Different findings of bispecific T cell engagers as well as CARs suggest cytokines secreted upon target cell ligation to cause the lysis of antigen-negative tumor cells in close proximity to the antigen-specific engagement (Slaney et al., 2018, Cancer Discov, 8 (8): 924-34). Next, the cytokine secretion profile of D2AP11-TCE was examined. Incubation of OVCAR3-FSHR target cells+human PBMCs with D2AP11-TCE led to the significant induction of IFN-γ, 237 soluble Fas (sFas), granzyme A, granzyme B and perforin compared to empty vector control at 48 hours and this construct drives robust tumor antigen specific killing (FIG. 19A). These effector cytokines are known to possess potential to change the tumor microenvironment and to induce endogenous antitumor immunity (Slaney et al., 2018, Cancer Discov. 8 (8): 924-34).

### FSHR Targeted T Cell Engager Decreased Tumor Burden In Vivo

To evaluate the in vivo antitumor effects of D2AP11-TCE, an in vivo challenge model was developed. For this model NSG mice were used which were administered K562 cells or FSHR overexpressing K562 cells (K562-FSHR) (FIG. 19B). The mice were inoculated with DNA encoded D2AP11-TCE (100 μg) and human T cells as described. Interestingly there were tumor escapes in K562 challenged mice in both D2AP11-TCE treated or empty vector control groups (FIG. 19C), whereas there was a significant reduction in tumor volume in the D2AP11-TCE treated group compared to empty vector control in K562-FSHR challenged group (FIG. 19D). After confirming the specificity and potency of D2AP11-TCE in this model, its effect in OVCAR3-FSHR challenged ovarian tumor mice model was examined. 14 days after tumor implantation, they were administered as DNA encoded D2AP11-TCE (100 μg), or empty vector (100 μg), twice two weeks apart for in vivo antibody generation. On day 14, they were inoculated with human T cells (10 million/mouse), and tumor volumes were measured periodically (FIG. 19E). Treatment using this bispecific led to significantly decreased tumor burden in OVCAR3-FSHR tumor bearing mice, while no similar impact was observed in the control group (FIG. 19F) supporting the potential of this novel approach for therapeutic development against OC.

### Example 3: Immune Checkpoint Inhibitors in Ovarian Cancer

Immune checkpoint inhibitors (CPIs) have dramatically improved survival of several cancers and under evaluation in Ovarian Cancer (OC). Unfortunately, CPIs have shown globally unsatisfactory results in OC.

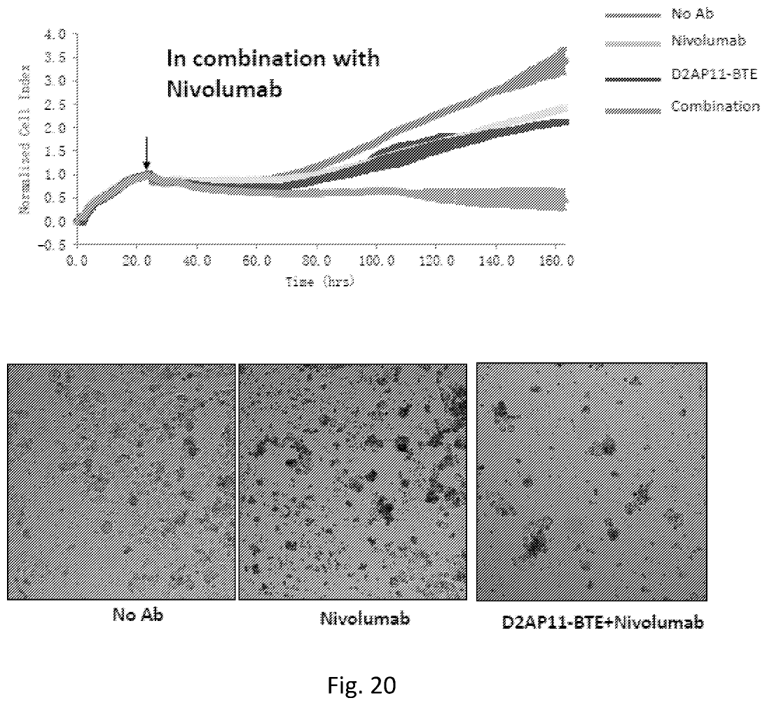

FIG. 20 shows that D2AP11-BTE synergistically enhances the killing by Nivolumab (anti-PD1 antibody) in ovarian cancer cells.

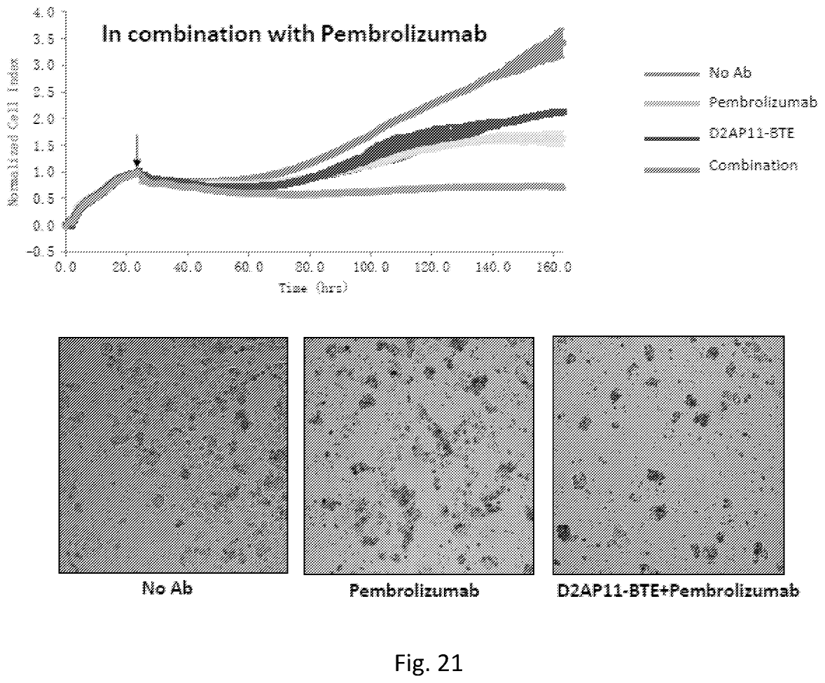

FIG. 21 shows that D2AP11-BTE synergistically enhances the killing by Pembrolizumab (anti-PD1 antibody) in ovarian cancer cells.

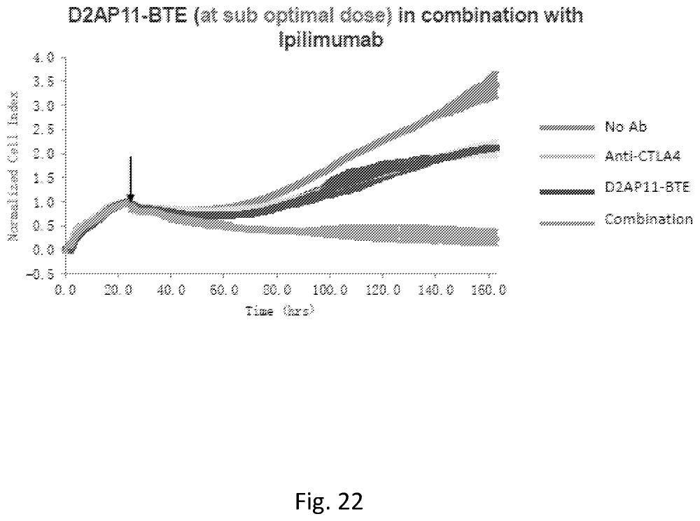

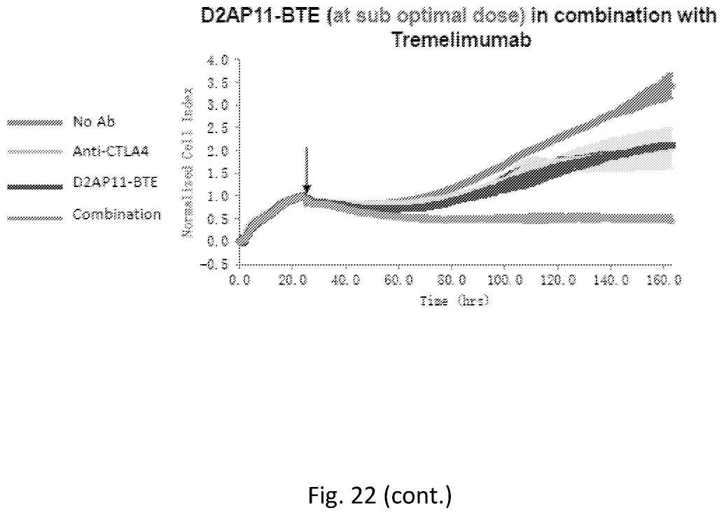

FIG. 22 shows that D2AP11-BTE synergistically enhances the killing by anti-CTLA4 antibodies in ovarian cancer cells.

### Example 4: CAR Molecules

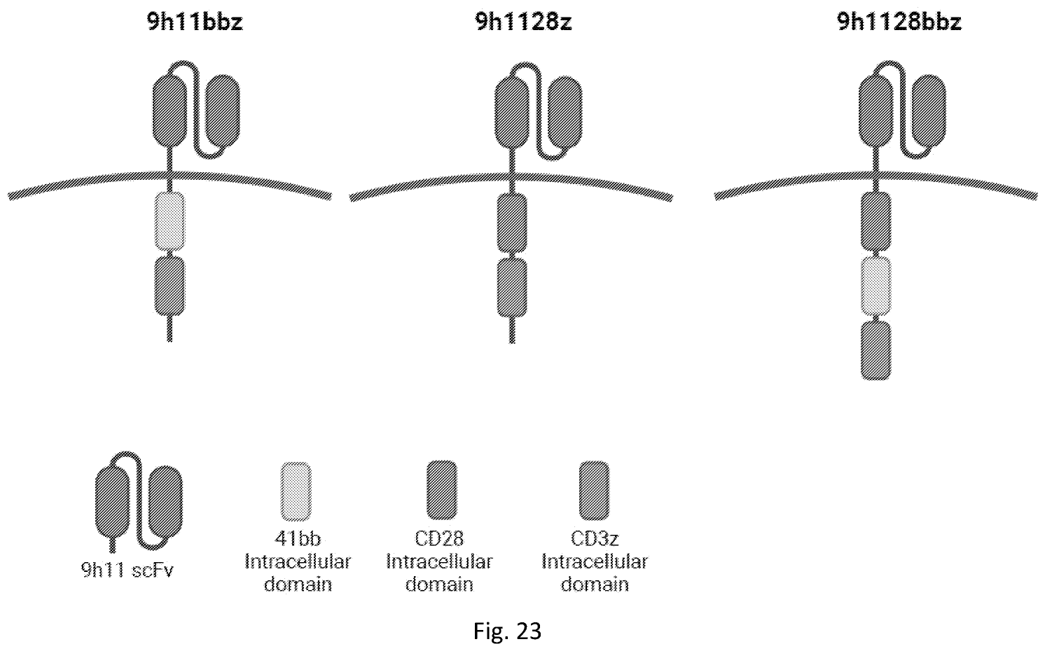

FIG. 23 shows the development of CART cells based off the 9h11 (D2AP11) clone targeting FSHR. The CART cells express an scFv targeting FSHR linked to a transmembrane domain and at least one intracellular domain (41bb, CD28 or CD3%).

Sequences

hu9h11xCD3 BTE (also referred to as D2AP11)
SEQ ID NO: 1-DNA sequence
ATGGATTGGACATGGATTCTGTTCCTGGTCGCAGCAGCCACAAGAGTGCATTCCGATATTCAGAT
GACCCAGTCCCCTTCAAGCCTGTCCGCCTCTGTGGGCGATAGGGTGACCATCAGCTGCAGAGCC
AGCGAGTCCGTGGACAACTACGGCATCTCCTTCCTGAATTGGTTTCAGCAGAAGCCCGGCAAGG
CCCCTAAGCTGCTGATCTATGCAGCAAGCAACCAGCGGTCCGGAGTGCCATCTCGCTTCTCTGGA
AGCGGATCCGGAACCGACTTCACCCTGACAATCAGCTCCCTGCAGCCCGAGGACTTCGCCACAT
ACTTTTGCCAGCAGAGCAAGGAGGTGCCTTGGACCTTCGGCCAGGGCACAAAGGTGGAGATCAA
GAGGGGCGGCGGCGGCAGCGGCGGCGGCGGCAGCGGCGGCGGCGGCTCTGAGGTGCAGCTGGT
GGAGAGCGGCGGCGGCCTGGTGCAGCCTGGCGGCTCTCTGAGGCTGTCTTGTAGCTTCTCCGGCT
TTTCTCTGAGCACCTCCGGAATGGGAGTGGGATGGATCAGACAGGCACCAGGCAAGGGCCTGGA
GTGGGTGGCCCACATCTGGTGGGACGATGACAAGCGGTACAACCCCGCCCTGAAGTCTCGCTTC
ACCCTGAGCGTGGATAGGTCCAAGAACACACTGTATCTGCAGATGAACAGCCTGAGAGCCGAGG
ACACCGCCACATACTATTGCGTGCAGATCAACTACGGCAATTATCGGTTTGATAATTGGGGCCAC
GGCACCCTGGTGACAGTGTCTAGCGGCGGCGGCGGCTCCGAAGTCCAGCTGGTCGAAAGCGGCG
GCGGCCTGGTCCAGCCAGGCGGCTCTCTGAGACTGAGCTGTGCAGCATCCGGATACTCTTTCACC
GGCTATACAATGAATTGGGTGAGGCAGGCACCTGGCAAGGGCCTGGAATGGGTGGCCCTGATCA
ACCCATACAAGGGCGTGAGCACCTATAATCAGAAGTTCAAGGATAGGTTTACCATCTCCGTGGA
CAAGTCTAAGAACACAGCCTACCTGCAAATGAACAGCTTACGCGCCGAGGACACAGCCGTGTAC
TATTGCGCCAGGAGCGGCTACTATGGCGATTCCGACTGGTATTTTGACGTGTGGGGCCAGGGCA
CCCTGGTCACAGTGTCCAGCGGCGGCGGCGGCAGCGGCGGCGGCGGCAGCGGCGGCGGCGGCT
CGGACATCCAGATGACCCAGAGCCCAAGCTCCCTGTCTGCCAGCGTCGGCGATAGGGTCACCAT
CACATGTAGAGCCTCCCAGGACATCCGGAACTACCTGAATTGGTATCAGCAGAAGCCAGGCAAG
GCCCCCAAGCTGCTGATCTACTATACAAGCAGACTGGAGTCCGGCGTGCCTTCTCGGTTCTCCGG
ATCTGGAAGCGGAACCGATTACACCCTGACAATCTCTAGCCTGCAGCCAGAGGACTTTGCCACA
TACTATTGTCAGCAGGGGAATACTCTGCCTTGGACCTTCGGACAGGGAACAAAAGTGGAAATCA
AATCCTCA
SEQ ID NO: 2-Amino Acid Sequence
MDWTWILFLVAAATRVHSDIQMTQSPSSLSASVGDRVTISCRASESVDNYGISFLN
WFQQKPGKAPKLLIYAASNQRSGVPSRFSGSGSGTDFTLTISSLQPEDFATYFCQQSKEVPWTFGQGT
KVEIKRGGGGSGGGGSGGGGSEVQLVESGGGLVQPGGSLRLSCSFSGFSLSTSGMGVGWIRQAP
GKGLEWVAHIWWDDDKRYNPALKSRFTLSVDRSKNTLYLQMNSLRAEDTATYYCVQINYGNY
RFDNWGHGTLVTVSSGGGGSEVQLVESGGGLVQPGGSLRLSCAASGYSFTGYTMNWVRQAPGKG
LEWVALINPYKGVSTYNQKFKDRFTISVDKSKNTAYLQMNSLRAEDTAVYYCARSGYYGDSDWYF
DVWGQGTLVTVSSGGGGSGGGGSGGGGSDIQMTQSPSSLSASVGDRVTITCRASQDIRNYLNWYQQ
KPGKAPKLLIYYTSRLESGVPSRFSGSGSGTDYTLTISSLQPEDFATYYCQQGNTLPWTFGQGTKVEIK
SS**
SEQ ID NO: 3-FSHR ScFV-no IgE leader sequence
EVQLVESGGGLVQPGGSLRLSCSFSGFSLSTSGMGVGWIRQAPGKGLEWVAHI
WWDDDKRYNPALKSRFTLSVDRSKNTLYLQMNSLRAEDTATYYCVQINYGNYRFDNWGHGT
LVTVSSGGGGSGGGGSGGGGSDIQMTQSPSSLSASVGDRVTISCRASESVDNYGISFLNWFQQKPGK
APKLLIYAASNQRSGVPSRFSGSGSGTDFTLTISSLQPEDFATYFCQQSKEVPWTFGQGTKVEIK
hu9h11bbz CART construct (SEQ ID NO: 4):
MDWTWILFLVAAATRVHSEVQLVESGGGLVQPGGSLRLSCSFSGFSLSTSGMG
VGWIRQAPGKGLEWVAHIWWDDDKRYNPALKSRFTLSVDRSKNTLYLQMNSLRAEDTATYY
CVQINYGNYRFDNWGHGTLVTVSSGGGGSGGGGSGGGGSDIQMTQSPSSLSASVGDRVTISCRASE
SVDNYGISFLNWFQQKPGKAPKLLIYAASNQRSGVPSRFSGSGSGTDFTLTISSLQPEDFATYFCQQSK
EVPWTFGQGTKVEIKTTTPAPRPPTPAPTIASQPLSLRPEACRPAAGGAVHTRGLDFACDIYIWAPLAG
TCGVLLLSLVITLYCNHRNKRGRKKLL YIFKQPFMRPVQTTQEEDGCSCRFPEEEEGGCELRVKFSRS
ADAPAYQQGQNQLYNELNLGRREEYDVLDKRRGRDPEMGGKPQRRKNPQEGLYNELQKDKMAEA
YSEIGMKGERRRGKGHDGLYQGLSTATKDTYDALHMQALPPR**
hu9h1128z CART construct (SEQ ID NO: 5):
MDWTWILFLVAAATRVHSEVQLVESGGGLVQPGGSLRLSCSFSGFSLSTSGMG
VGWIRQAPGKGLEWVAHIWWDDDKRYNPALKSRFTLSVDRSKNTLYLQMNSLRAEDTATYY
CVQINYGNYRFDNWGHGTLVTVSSGGGGSGGGGSGGGGSDIQMTQSPSSLSASVGDRVTISCRASE
SVDNYGISFLNWFQQKPGKAPKLLIYAASNQRSGVPSRFSGSGSGTDFTLTISSLQPEDFATYFCQQSK
EVPWTFGQGTKVEIKTTTPAPRPPTPAPTIASQPLSLRPEACRPAAGGAVHTRGLDFACDIYIWAPLAG
TCGVLLLSLVITLYCNHRNRSKRSRLLHSDYMNMTPRRPGPTRKHYQPYAPPRDFAAYRSRVKFSRS
ADAPAYQQGQNQLYNELNLGRREEYDVLDKRRGRDPEMGGKPQRRKNPQEGLYNELQKDKMAEA
YSEIGMKGERRRGKGHDGLYQGLSTATKDTYDALHMQALPPR**
hu9h1128bbz CART construct (SEQ ID NO: 6):
MDWTWILFLVAAATRVHSEVQLVESGGGLVQPGGSLRLSCSFSGFSLSTSGMG
VGWIRQAPGKGLEWVAHIWWDDDKRYNPALKSRFTLSVDRSKNTLYLQMNSLRAEDTATYY
CVQINYGNYRFDNWGHGTLVTVSSGGGGSGGGGSGGGGSDIQMTQSPSSLSASVGDRVTISCRASE
SVDNYGISFLNWFQQKPGKAPKLLIYAASNQRSGVPSRFSGSGSGTDFTLTISSLQPEDFATYFCQQSK
EVPWTFGQGTKVEIKTTTPAPRPPTPAPTIASQPLSLRPEACRPAAGGAVHTRGLDFACDIYIWAPLAG
TCGVLLLSLVITLYCNHRNRSKRSRLLHSDYMNMTPRRPGPTRKHYQPYAPPRDFAAYRSKRGRKK
LLYIFKQPFMRPVQTTQEEDGCSCRFPEEEEGGCELRVKFSRSADAPAYQQGQNQLYNELNLGRREE
YDVLDKRRGRDPEMGGKPQRRKNPQEGLYNELQKDKMAEAYSEIGMKGERRRGKGHDGLYQGLS
TATKDTYDALHMQALPPR**

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

What is claimed is:

1. A synthetic bispecific immune cell engager (BICE), wherein the synthetic bispecific immune cell engager comprises at least one least one follicle stimulating hormone receptor (FSHR) antigen binding domain, and at least one immune cell engaging domain.

2. The BICE of claim 1, wherein the immune cell engaging domain targets a cell selected from the group consisting of a T cell, an antigen presenting cell, a natural killer (NK) cell, a neutrophil and a macrophage.

3. The BICE of claim 1, wherein the immune cell engaging domain targets at least one T cell specific receptor molecule selected from the group consisting of CD3, the T cell receptor (TCR), CD28, CD16, NKG2D, Ox40, 4-1BB, CD2, CD5, CD40, FcgRs, FceRs, FcaRs and CD95.

4. The BICE of claim 3, wherein the immune cell engaging domain targets CD3.

5. The BICE of claim 1 comprising one or more sequences selected from the group consisting of:
a) an amino acid sequence having at least about 90% identity over an entire length of the amino acid sequence to SEQ ID NO:2;
b) a fragment of an amino acid sequence having at least about 90% identity over at least 65% of SEQ ID NO:2;
c) an amino acid sequence of SEQ ID NO:2; and
d) a fragment of an amino acid sequence comprising at least 65% of an amino acid sequence of SEQ ID NO:2.

6. A nucleic acid molecule encoding a BICE of any one of claims 1-5.

7. The nucleic acid molecule of claim 6, wherein the nucleic acid molecule is selected from the group consisting of an RNA molecule and a DNA molecule.

8. The nucleic acid molecule of claim 6 or claim 7, comprising a nucleotide sequence selected from the group consisting of:
a) a nucleotide sequence having at least about 90% identity over an entire length of the nucleic acid sequence to SEQ ID NO:1;
b) a fragment of a nucleotide sequence having at least about 90% identity over at least 65% of the nucleic acid sequence to SEQ ID NO:1;
c) a nucleotide sequence of SEQ ID NO:1; and
d) a fragment of a nucleotide sequence comprising at least 65% of a nucleotide sequence of SEQ ID NO:1.

9. The nucleic acid molecule of any one of claims 6-8, wherein the nucleic acid molecule comprises an expression vector.

10. A composition comprising the BICE of any one of claims 1-5 or the nucleic acid molecule of any one of claims 6-9.

11. The composition of claim 10, further comprising a pharmaceutically acceptable excipient.

12. The composition of claim 10, further comprising at least one immune checkpoint inhibitor.

13. The composition of claim 10, further comprising at least one nucleic acid molecule encoding at least one immune checkpoint inhibitor.

14. The composition of claim 12 or 13, wherein the immune checkpoint inhibitor is selected from the group consisting of an inhibitor of PD-1, an inhibitor of PD-L-1, an inhibitor of cytotoxic T-lymphocyte antigen-4 (CTLA-4), an inhibitor of mucin-domain containing-3 (TIM-3), and an inhibitor of Lymphocyte Activating 3 (LAG3).

15. The composition of claim 10, wherein the composition comprises a lipid nanoparticle comprising the BICE of any one of claims 1-5 or the nucleic acid molecule of any one of claims 6-9.

16. A method of preventing or treating a disease or disorder associated with FSHR expression in a subject, the method comprising administering to the subject the BICE of any one of claims 1-5, the nucleic acid molecule of any one of claims 6-9 or the composition of any one of claims 10-15.

17. The method of claim 16, wherein the disease is selected from the group consisting of a benign tumor, cancer and a cancer-associated disease.

18. The method of claim 17, wherein the disease is selected from the group consisting of ovarian cancer, breast cancer, prostate cancer, renal cancer, colo-rectal cancer, stomach cancer, lung cancer, testicular cancer, endometrial cancer, and thyroid cancer.

19. An FSHR scFv molecule comprising a sequence selected from the group consisting of:
a) an amino acid sequence having at least about 90% identity over the entire length of the amino acid sequence of SEQ ID NO:3;
b) a fragment of an amino acid sequence having at least about 90% identity over at least 65% of SEQ ID NO:3;
c) an amino acid sequence of SEQ ID NO:3; and
d) a fragment of an amino acid sequence comprising at least 65% of the amino acid sequence of SEQ ID NO:3.

20. A composition comprising the FSHR scFv of claim 19.

21. The composition of claim 20, further comprising a pharmaceutically acceptable excipient.

22. The composition of claim 21, further comprising at least one immune checkpoint inhibitor.

23. The composition of claim 22, wherein the immune checkpoint inhibitor is selected from the group consisting of an inhibitor of PD-1, an inhibitor of PD-L-1, an inhibitor of cytotoxic T-lymphocyte antigen-4 (CTLA-4), an inhibitor of mucin-domain containing-3 (TIM-3), and an inhibitor of Lymphocyte Activating 3 (LAG3).

24. The composition of claim 20, wherein the composition comprises a lipid nanoparticle comprising an scFv of claim 19.

25. The composition of claim 20, wherein the composition comprises a CAR molecule comprising an scFv of claim 19.

26. The composition of claim 25, wherein the CAR molecule comprises a sequence selected from the group consisting of SEQ ID NO:4, SEQ ID NO:5 and SEQ ID NO: 6.

27. The composition of claim 20, comprising a cell expressing the scFv of claim 19 or the CAR molecule of any one of claims 25-26.

28. An FSHR-specific chimeric antigen receptor (CAR) molecule.

29. The CAR molecule of claim 28, wherein the CAR comprises an FSHR specific ScFv comprising a nucleotide sequence as set forth in SEQ ID NO:3.

30. The CAR molecule of claim 28, wherein the CAR comprises a sequence selected from the group consisting of SEQ ID NO:4, SEQ ID NO:5 and SEQ ID NO: 6.

31. A composition comprising an FSHR-specific chimeric antigen receptor (CAR) molecule of any one of claims 28-30.

32. The composition of claim 31, wherein the composition comprises a cell expressing the FSHR-specific chimeric antigen receptor (CAR) molecule.

33. The composition of claim 32, wherein the cell is an engineered T cell.

34. A method of preventing or treating a disease or disorder associated with FSHR expression in a subject, the method comprising administering to the subject an scFv antibody fragment of claim 19, a composition of any one of claims 20-27, a CAR molecule of any one of claims 28-30 or a composition of any one of claims 31-33.

35. The method of claim 34, wherein the disease is selected from the group consisting of a benign tumor, cancer and a cancer-associated disease.

36. The method of claim 35, wherein the disease is selected from the group consisting of ovarian cancer, breast cancer, prostate cancer, renal cancer, colo-rectal cancer, stomach cancer, lung cancer, testicular cancer, endometrial cancer, and thyroid cancer.
